(19) United States
(12) Patent Application Publication
(10) Pub. No.: US 2011/0124070 A1
(43) Pub. Date: May 26, 2011

(54) PROCESS FOR ALCOHOL AND CO-PRODUCT PRODUCTION FROM GRAIN SORGHUM

(76) Inventors: Gang Duan, Shanghai (CN); Jayarama K. Shetty, Pleasanton, CA (US); Hongxian Xu, Jiangsu (CN)

(21) Appl. No.: 12/994,319

(22) PCT Filed: May 29, 2009
(86) PCT No.: PCT/US09/45594

§ 371 Date: Jan. 26, 2011

Related U.S. Application Data
(60) Provisional application No. 61/130,187, filed on May 29, 2008.

(51) Int. Cl. C12P 7/06

(52) U.S. Cl. 435/161

(57) ABSTRACT

Described herein are methods for producing alcohol and particularly ethanol from milled sorghum.

### CROSS REFERENCE

The present application claims priority to U.S. Provisional Patent Application Ser. No. 61/130,187 filed May 29, 2008, which is hereby incorporated by reference in its entirety.

### FIELD OF THE INVENTION

The present invention relates to no-cook methods for producing alcohol (e.g., ethanol) from fermentations using sorghum as a feedstock. The methods comprise using phytase enzymes, starch hydrolyzing enzymes having granular starch hydrolyzing activity and non-starch polysaccharide hydrolyzing enzymes in the no-cook process.

### BACKGROUND OF THE INVENTION

The use of renewable energy such as biofuels is gaining importance due to the shortage and expense of petroleum products. As a result, the biofuel ethanol market has been growing by double-digits over the last few years and that trend is expected to continue over at least the next three to five years. One problem with the use of fermentation ethanol for energy is that the method is energy-consuming and therefore, the efficiency is still in need of improvement. Another problem is that with the increased use of fermentation ethanol, more side-products are produced such as distillers dried grains and solubles (DDGS). For example, one metric ton of corn kernel generally produces around 300 Kgs of DDGS in a dry milling process. So, the increase in ethanol production to meet rapidly growing market needs also results in an increase in the volume of DDGS. While the DDGS can be used in animal feed formulations, they typically have high levels of phytic acid which reduces the number of animals that can digest the DDGS and increases pollution problems when digested.

A number of agricultural crops present themselves as viable candidates for the conversion of starch to glucose to produce a variety of biochemicals, including renewable biofuels, like ethanol. Corn is the most widely used starch-based fermentation feedstock for the production of ethanol, but other high-starch content grains like sorghum and rice are beginning to be considered as viable feedstock in the production of ethanol.

In general, alcohol fermentation processes and particularly ethanol production processes include wet milling or dry milling processes. Reference is made to Bothast et al., 2005, Appl. Microbiol. Biotechnol. 67:19-25 and THE ALCOHOL TEXTBOOK, 3^{rd} Ed (K. A. Jacques et al. Eds) 1999 Nottingham University Press, UK for a review of these processes.

In general, dry milling involves a number of basic steps, which include: grinding, cooking, liquefaction, saccharification, fermentation and separation of liquid and solids to produce alcohol and other co-products. Generally, whole cereal grain, such as corn, is ground to a fine particle size and then mixed with liquid in a slurry tank. The slurry is subjected to high temperatures in a jet cooker along with liquefying enzymes (e.g. alpha-amylases) to hydrolyze the starch in the cereal to dextrins. The mixture is cooled down and further treated with saccharifying enzymes (e.g. glucoamylases) to produce fermentable glucose. The mash containing glucose is then fermented for approximately 24 to 120 hours in the presence of ethanol producing microorganisms. The solids in the mash are separated from the liquid phase and ethanol and useful co-products such as distillers' grains are obtained.

More recently, processes have been introduced which eliminate the cooking step or which reduce the need for treating cereal grains at high temperatures. These processes which are sometimes referred to as no-cook, low temperature or warm cook, include milling of a cereal grain and combining the ground cereal grain with liquid to form a slurry which is then mixed with one or more enzymes having granular starch hydrolyzing activity and optionally yeast at temperatures below the granular starch gelatinization temperature to produce ethanol and other co-products (U.S. Pat. No. 4,514,496, WO 03/066826; WO 04/081193; WO 04/106533; WO 04/080923 and WO 05/069840).

While these processes offer certain improvements over previous processes, additional process improvements are needed by the industry for the conversion of grain sorghum which results in higher carbon conversion and energy efficiency and high alcohol production.

### SUMMARY OF THE INVENTION

In some embodiments, the invention relates to a method of producing alcohol from milled sorghum comprising, contacting a slurry comprising milled sorghum having a dry solids (ds) content of between 20 to 50% w/w with at least one phytase, at least one alpha amylase (AA), at least one glucoamylase (GA), at least one non-starch polysaccharide hydrolyzing enzyme and a fermentation organism at a temperature below the starch gelatinization temperature of the sorghum, at a pH of about 3.5 to about 7.0 for about 10 to about 250 hours, wherein said at least one AA and/or at least one GA has granular starch hydrolyzing activity and producing alcohol. In some aspects of this embodiment, the alcohol is ethanol; the at least one non-starch polysaccharide hydrolyzing enzyme is selected from: cellulases, beta-glucosidases, pectinases, xylanases, beta-glucanases, hemicellulases or a combination thereof. In some aspects of this embodiment the phytase, alpha amylase, glucoamylase, and non-starch polysaccharide hydrolyzing enzyme are added as an enzyme blend. In other aspects of this embodiment, the method further comprises contacting the slurry with at least one protease. The protease may be an acid fungal protease. The acid fungal protease may be derived from a Trichoderma sp. In additional aspects of this embodiment, the acid fungal protease is added at a concentration of between about 1 ppm and about 10 ppm. In yet further aspects of this embodiment, the method further comprises contacting the slurry with at least a second non-starch polysaccharide hydrolyzing enzyme. In still other aspects of this embodiment, the contacting is at a temperature of between 20° C. to 80° C. also between 25° C. and 40° C. In other aspects, the contacting is at a temperature of between 55° C. to 77° C. and then reduced to between 25° C. to 35° C. before the yeast is added. In other aspects of this embodiment, the phytase supplied in the contacting step is from about 0.01 to about 10.0 FTU/g ds, also from about 0.1 to about 5.0 FTU/g ds, and from about 1 to about 4 FTU/g ds. In yet other aspects of this embodiment, the slurry comprises grain sorghum in admixture with at least one other grain selected from corn, wheat, rye, barley, rice or combinations thereof.

In some embodiments, the invention relates to a process for producing ethanol from sorghum, comprising, obtaining a slurry of milled sorghum, contacting the slurry with a combination of enzymes comprising a phytase, an alpha amylase, a glucoamylase, and a non-starch polysaccharide hydrolyzing enzyme at a temperature below the gelatinization temperature of sorghum to produce fermentable sugars; and fermenting the fermentable sugars in the presence of a fermenting microorganism at a temperature of between 10° C. and 40° C. for a period of 10 hours to 250 hours, and producing ethanol, wherein the yield of ethanol is increases relative to a comparable process using only an alpha amylase and a glucoamylase. In some aspects the ethanol yield will be at least 8%, at least 10%, least 12%, at least 14% and at least 16%, v/v. In other aspects the yield of ethanol will be increased between at least 1% and at least 10%. In some aspect the contacting step is conducted at a temperature of between 45° C. and 65° C. In further aspects, the process comprises reducing the temperature after the contacting step.

In some embodiments, the invention relates to methods of producing ethanol from sorghum comprising, contacting a slurry comprising granular starch from grain sorghum with at least one phytase, at least one alpha amylase (AA), at least one glucoamylase (GA) at least one non-starch polysaccharide hydrolyzing enzyme, at least one acid fungal protease and a fermentation organism for a time sufficient to produce ethanol, wherein said at least one AA and/or at least one GA is a granular starch hydrolyzing enzyme (GSHE), at a temperature below the starch gelatinization temperature of the grain, wherein said non-starch polysaccharide hydrolyzing enzymes are chosen from: a cellulase, a xylanase, a pectinase, a beta-glucosidase, a beta-glucanase and/or a hemicellulase.

### DETAILED DESCRIPTION OF THE INVENTION

Methods of the invention involve the use of non-starch polysaccharide hydrolyzing enzymes in combination with phytases and granular starch hydrolyzing enzymes (GSHE) to increase the ethanol yield in no-cook fermentations using sorghum. Using conventional processes, the yield of ethanol from sorghum is typically very low. While there are a number of factors contributing to the low yield, the high concentration of tannins in sorghum is one contributing factor. This is because, when heated, tannins cross-link with proteins, starches and other molecules creating a web-like structure. The cross-linking makes starch within the sorghum inaccessible to enzymes and results in a loss of fermentable sugars. Thus, the use of a no-cook process increases accessibility of the starch and results in better fermentation efficiency with the result that the ethanol yield increases. The methods also have the advantage of providing nutrients and/or growth factors for yeast by hydrolyzing the phytic acid to inositol (a nutrient for yeast) and phosphate (a nutrient for both yeast and feed animals). This also results in an increased fermentation efficiency.

Methods of the invention comprise contacting sorghum with a fermenting organism in a no-cook process and with the following enzymes simultaneously or separately: at least one alpha amylase, at least one glucoamylase, wherein said alpha amylase and/or glucoamylase is a granular starch hydrolyzing enzyme (GSHE), at least one phytase, and at least one non-starch polysaccharide hydrolyzing enzyme (e.g., cellulases, hemi-cellulases, beta glucosidases, beta glucanase, xylanase and/or pectinases) to produce alcohol. The methods can also comprise adding secondary enzymes such as acid fungal proteases. The no-cook process can be conducted at a temperature below the starch gelatinization temperature of sorghum. In some embodiments, the method is conducted at a temperature conducive to yeast fermentation. In some embodiments the contacting occurs as a pretreatment. In some embodiments, the contacting, fermentation and/or pretreatment occurs at a temperature below the starch gelatinization temperature of granular starch in the sorghum. In some embodiments, the pretreatment occurs at a temperature below the gelatinization temperature of the granular starch in the sorghum, but at a temperature closer to the optimal temperature for the non-starch polysaccharide hydrolyzing enzymes and/or other enzymes used in the process. The process results in increased ethanol yield, increased fermentation efficiency and/or a reduced amount of phytic acid in the DDGS as compared to substantially similar methods conducted without addition of the phytase and non-starch polysaccharide hydrolyzing enzymes.

Thus, embodiments of the process include compositions and methods of contacting sorghum with an enzyme composition comprising at least one phytase, at least one alpha amylase, and at least one glucoamylase, wherein said alpha amylase and/or glucoamylase is a granular starch hydrolyzing enzyme and at least one non-starch polysaccharide hydrolyzing enzyme (e.g., cellulases, hemi-cellulases, xylanase, beta glucosdases, beta glucanase, and/or pectinases) during fermentation at a temperature and for a time sufficient to produce ethanol. The methods result in an increased ethanol yield, increased fermentation efficiency and/or a reduction in the amount of phytic acid in the DDGS. In some embodiments, the at least one non-starch polysaccharide hydrolyzing enzyme are chosen from: cellulases, hemicellulases, xylanase, beta glucanases, beta-glucosidases, and pectinases. The methods can also comprise the addition of an acid fungal protease. In some embodiments, the method involves incubating and/or fermenting sorghum at a temperature conducive to fermentation by the fermentation organism (e.g., 28-38° C.) at a pH between about 3.5 and 7.0 and for between 10 and 250 hours.

### DEFINITIONS

Unless otherwise indicated, the practice of the invention involves conventional techniques commonly used in molecular biology, protein engineering, recombinant DNA techniques, microbiology, cell biology, cell culture, transgenic biology, immunology, and protein purification, which are within the skill of the art. Such techniques are known to those of skill in the art and are described in numerous texts and reference works. All patents, patent applications, articles and publications mentioned herein, both supra and infra, are hereby expressly incorporated herein by reference.

Unless defined otherwise herein, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described. Accordingly the terms defined immediately below are more fully described by reference to the Specification as a whole. Also, as used herein, the singular “a”, “an” and “the” includes the plural reference unless the context clearly indicates otherwise. Numeric ranges are inclusive of the numbers defining the range. Thus, for example, reference to a composition containing “a compound” includes a mixture of two or more compounds. It should also be noted that the term “or” is generally employed in its sense including “and/or” unless the content clearly dictates otherwise. Unless otherwise indicated amino acids are written left to right in amino to carboxy orientation, respectively. It is to be understood that this invention is not limited to the particular methodology, protocols, and reagents described as these may vary, depending upon the context they are used by those of skill in the art. Furthermore, the headings provided herein are not limitations of the various aspects or embodiments of the invention which can be had by reference to the specification as a whole. Accordingly the terms defined immediately below are more fully defined by reference to the specification as a whole. Nonetheless, in order to facilitate understanding of the invention, a number of terms are defined below. Other features and advantages of the invention will be apparent from the present specification and claims.

As used herein, the term “starch” refers to any material comprised of the complex polysaccharide carbohydrates of plants, comprised of amylose and amylopectin with the formula (C₆H₁₀O₅), wherein x can be any number.

As used herein, the term “granular starch” and refers to raw (uncooked) starch, that is starch in its natural form found in plant material (e.g. grains and tubers).

As used herein, the term “granular starch substrate” refers to a substance containing granular starch.

As used herein, the term “dry solids content (DS)” refers to the total solids of a slurry in % on a dry weight basis.

As used herein, the term “slurry” refers to an aqueous mixture comprising insoluble solids, (e.g. granular starch).

As used herein, the term “oligosaccharides” refers to any compound having 2 to 10 monosaccharide units joined in glycosidic linkages. These short chain polymers of simple sugars include dextrins.

As used herein, the term “soluble starch” refers to starch which results from the hydrolysis of insoluble starch (e.g. granular starch).

As used herein, the term “mash” refers to a mixture of a fermentable substrate in liquid used in the production of a fermented product and is used to refer to any stage of the fermentation from the initial mixing of the fermentable substrate with one or more starch hydrolyzing enzymes and fermenting organisms through the completion of the fermentation run.

As used herein, the terms “saccharifying enzyme” and “starch hydrolyzing enzymes” refer to any enzyme that is capable of converting starch to mono- or oligosaccharides (e.g. a hexose or pentose).

As used herein, the terms “granular starch hydrolyzing (GSH) enzyme” and “enzymes having granular starch hydrolyzing (GSH) activity” refer to enzymes, which have the ability to hydrolyze starch in granular form.

As used herein, the term “non-starch polysaccharide hydrolyzing enzymes” are enzymes capable of hydrolyzing complex carbohydrate polymers such as cellulose, hemicellulose, and pectin. For example, cellulases (endo and exo-glucanases, beta glucosidase) hemicellulases (xylanases) and pectinases are non-starch polysaccharide hydrolyzing enzymes.

As used herein, the term “hydrolysis of starch” refers to the cleavage of glucosidic bonds with the addition of water molecules.

As used herein, the term “alpha-amylase (e.g., E.C. class 3.2.1.1)” refers to enzymes that catalyze the hydrolysis of alpha-1,4-glucosidic linkages These enzymes have also been described as those effecting the exo or endohydrolysis of 1,4-α-D-glucosidic linkages in polysaccharides containing 1,4-α-linked D-glucose units.

As used herein, the term “gelatinization” means solubilization of a starch molecule by cooking to form a viscous suspension.

As used herein, the term “gelatinization temperature” refers to the temperature at which gelatinization of a starch containing substrate begins. In some embodiments, this is lowest temperature at which gelatinization begins. The exact temperature of gelatinization depends on the specific starch and may vary depending on factors such as plant species and environmental and growth conditions. The initial starch gelatinization temperature ranges for a number of granular starches, for example, include barley (52° C. to 59° C.), wheat (58° C. to 64° C.), rye (57° C. to 70° C.), corn (62° C. to 72° C.), high amylose corn (67° C. to 80° C.), rice (68° C. to 77° C.), sorghum (68° C. to 77° C.), potato (58° C. to 68° C.), tapioca (59° C. to 69° C.) and sweet potato (58° C. to 72° C.). (See, e.g., J. J. M. Swinkels pg 32-38 in STARCH CONVERSION TECHNOLOGY, Eds Van Beynum et al., (1985) Marcel Dekker Inc. New York and The Alcohol Textbook 3^{rd} ED. A Reference for the Beverage, Fuel and Industrial Alcohol Industries, Eds Jacques et al., (1999) Nottingham University Press, UK).

As used herein, the term “below the gelatinization temperature” refers to a temperature that is less than the gelatinization temperature.

As used herein, the term “no-cook” refers to the absence of heating to a temperature above the gelatinization temperature of a starch-containing substrate.

As used herein, the term “glucoamylase” refers to the amyloglucosidase class of enzymes (e.g., E.C.3.2.1.3, glucoamylase, 1,4-alpha-D-glucan glucohydrolase). These are exo-acting enzymes, which release glucosyl residues from the non-reducing ends of amylase and amylopectin molecules. The enzymes also hydrolyzes alpha-1,6 and alpha-1,3 linkages although at much slower rate than alpha-1,4 linkages.

As used herein, the phrase “simultaneous saccharification and fermentation (SSF)” refers to a process in the production of end-products in which a fermenting organism, such as an ethanol producing microorganism, and at least one enzyme, such as a saccharifying enzyme are combined in the same process step in the same vessel.

As used herein, the term “saccharification” refers to enzymatic conversion of a directly unusable polysaccharide to a mono- or oligosaccharide for fermentative conversion to an end-product.

As used herein, the term “milling” refers to the breakdown of cereal grains to smaller particles. In some embodiments the term is used interchangeably with grinding.

As used herein, the term “dry milling” refers to the milling of dry whole grain, wherein fractions of the grain such as the germ and bran have not been purposely removed.

As used herein, the term “liquefaction” refers to the stage in starch conversion in which gelatinized starch is hydrolyzed to give low molecular weight soluble dextrins.

As used herein, the term “thin-stillage” refers to the resulting liquid portion of a fermentation which contains dissolved material and suspended fine particles and which is separated from the solid portion resulting from the fermentation. Recycled thin-stillage in industrial fermentation processes is frequently referred to as “back-set”.

As used herein, the term “vessel” includes but is not limited to tanks, vats, bottles, flasks, bags, bioreactors and the like. In some embodiments, the term refers to any receptacle suitable for conducting the saccharification and/or fermentation processes encompassed by the invention.

As used herein, the term “end-product” refers to any carbon-source derived product which is enzymatically converted from a fermentable substrate. In some preferred embodiments, the end-product is an alcohol, such as ethanol.

As used herein the term “fermenting organism” refers to any microorganism or cell which is suitable for use in fermentation for directly or indirectly producing an end-product.

As used herein the term “ethanol producer” or ethanol producing microorganism” refers to a fermenting organism that is capable of producing ethanol from a mono- or oligosaccharide.

As used herein, the term “enzymatic conversion” in general refers to the modification of a substrate by enzyme action. The term as used herein also refers to the modification of a fermentable substrate, such as a granular starch containing substrate by the action of an enzyme.

The terms “recovered”, “isolated”, and “separated” as used herein refer to a compound, protein, cell, nucleic acid or amino acid that is removed from at least one component with which it is naturally associated.

As used herein, the term “yield” refers to the amount of end-product produced using the methods of the present invention. In some embodiments, the term refers to the volume of the end-product and in other embodiments, the term refers to the concentration of the end-product.

As used herein the term “fermentation efficiency” refers to the percent actual weight of alcohol produced compared to the theoretical weight of ethanol from glucose producing substrate i.e. starch actual using the following formula as described (Yeast to Ethanol, 1993, 5, 2^{nd} edition, 241-287, Academic Press, Ltd.). The total starch content on a dry weight basis, conversion of starch to fermentable sugars by enzymatic hydrolysis during fermentation and chemical grain from starch to glucose is taken into consideration.

$$\% Fermentation Efficiency = \frac{Weight of ethanol produced \times 100}{Theoretical weight of ethanol from produced glucose}$$

As used herein, the term “DE” or “dextrose equivalent” is an industry standard for measuring the concentration of total reducing sugars, calculated as D-glucose on a dry weight basis. Unhydrolyzed granular starch has a DE that is essentially 0 and D-glucose has a DE of 100. An instructive method for determining the DE of a slurry or solution is described in Schroorl's method (Fehling's assay titration).

As used herein, the “fermentable sugars” are sugars that can be directly digested by fermentation organisms (e.g. yeast, for example). Some examples of fermentable sugars include fructose, maltose, glucose, sucrose, and galactose.

As used herein, the “dextrins” are short chain polymers of glucose (e.g., 2 to 10 units). As used herein, the term “glucose syrup” refers to an aqueous composition containing glucose solids. Glucose syrup will have a DE of at least 20. In some embodiments, glucose syrup will not contain more than 21% water and will not contain less than 25% reducing sugar calculated as dextrose. In some embodiments, glucose syrup will include at least about 90% D-glucose and in another embodiment glucose syrup will include at least about 95% D-glucose. In some embodiments the terms glucose and glucose syrup are used interchangeably.

As used herein “fermentation feedstock” means the grains or cereals used in the fermentation as raw materials such as corn, sorghum, wheat, barley, rye, etc.

As used herein, the term “total sugar content” refers to the total sugar content present in a starch composition.

As used herein, the term “fermentation” refers to the enzymatic and anaerobic breakdown of organic substances by microorganisms to produce simpler organic compounds. While fermentation occurs under anaerobic conditions it is not intended that the term be solely limited to strict anaerobic conditions, as fermentation also occurs in the presence of oxygen.

As used herein, the term “derived” encompasses the terms “originated from”, “obtained” or “obtainable from”, and “isolated from” and in some embodiments as used herein means that a polypeptide encoded by the nucleotide sequence is produced from a cell in which the nucleotide is naturally present or in which the nucleotide has been inserted.

As used herein, the terms “recovered”, “isolated”, and “separated” as used herein refer to a protein, cell, nucleic acid or amino acid that is removed from at least one component with which it is naturally associated.

As used herein, the terms “protein” and “polypeptide” are used interchangeability herein. In the present disclosure and claims, the conventional one-letter and three-letter codes for amino acid residues are used. The 3-letter code for amino acids as defined in conformity with the IUPAC-IUB Joint Commission on Biochemical Nomenclature (JCBN). It is also understood that a polypeptide can be coded for by more than one nucleotide sequence due to the degeneracy of the genetic code.

As used herein, the term “contacting” refers to the placing of at least one enzyme in sufficiently close proximity to its respective substrate to enable the enzyme(s) to convert the substrate to at least one end-product. In some embodiments, the end-product is a “product of interest” (i.e., an end-product that is the desired outcome of the fermentation reaction). Those skilled in the art will recognize that mixing at least one solution comprising the at least one enzyme with the respective enzyme substrate(s) results in “contacting.”

The headings provided herein are not limitations of the various aspects or embodiments of the invention, which can be had by reference to the specification as a whole.

Although any methods and materials similar or equivalent to those described herein can be used in the practice or testing of the present invention, exemplary and preferred methods and materials are now described. All publications mentioned herein are incorporated herein by reference to disclose and describe the methods and/or materials in connection with which the publications are cited.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

The publications discussed herein are provided solely for their disclosure prior to the filing date of the present application. Nothing herein is to be construed as an admission that the present invention is not entitled to antedate such publication by virtue of prior invention.

### Exemplary Embodiments

The invention is directed to methods of increasing the alcohol yield in no-cook fermentation methods utilizing sorghum as a feedstock. Using conventional processes, the yield of ethanol from sorghum is typically very low. While there are a number of factors contributing to the low yield, the high concentration of tannins in sorghum contributes substantially. When heated, tannins cross-link with proteins, starches and other molecules creating a web-like structure. The cross-linking makes starch within the sorghum less accessible to enzymes and results in a loss of fermentable sugars. Thus, the use of a no-cook process increases accessibility of the starch and results in better fermentation efficiency with the result that the ethanol yield increases.

Methods of the invention comprise contacting mill sorghum with a fermenting organism and with the following enzymes simultaneously or separately: at least one alpha amylase, at least one glucoamylase, wherein said alpha amylase and/or glucoamylase is a granular starch hydrolyzing enzyme (GSHE), at least one phytase, and at least one non-starch polysaccharide hydrolyzing enzyme (e.g., cellulases, hemi-cellulases, beta glucosidases, beta glucanase, xylanase and/or pectinases) to produce alcohol. The methods can also comprise adding secondary enzymes such as acid fungal proteases. The no-cook process can be conducted at a temperature below the starch gelatinization temperature of sorghum. In some embodiments, the method is conducted at a temperature conducive to yeast fermentation. In some embodiments the contacting occurs as a pretreatment. In some embodiments, the contacting, fermentation and/or pretreatment occurs at a temperature below the starch gelatinization temperature of granular starch in the sorghum. In some embodiments, the pretreatment occurs at a temperature below the gelatinization temperature of the granular starch in the sorghum, but at a temperature closer to the optimal temperature for the non-starch polysaccharide hydrolyzing enzymes and/or other enzymes used in the process. The process results in increased ethanol yield, increased fermentation efficiency and/or a reduced amount of phytic acid in the DDGS as compared to substantially similar methods conducted without addition of the phytase and non-starch polysaccharide hydrolyzing enzymes.

Thus, embodiments of the process include compositions and methods of contacting sorghum with an enzyme composition comprising at least one phytase, at least one alpha amylase, and at least one glucoamylase, wherein said alpha amylase and/or glucoamylase is a granular starch hydrolyzing enzyme, and at least one non-starch polysaccharide hydrolyzing enzyme (e.g., cellulases, hemi-cellulases, beta glucosidases, beta glucanase, xylanase and/or pectinases). The methods result in an increased ethanol production and/or an increased fermentation efficiency and/or a reduction in the amount of phytic acid in the DDGS. In some embodiments, the at least one non-starch polysaccharide hydrolyzing enzyme is chosen from: cellulases, hemicellulases, xylanases, beta glucanases, beta-glucosidases, and pectinases. The methods can also comprise the addition of an acid fungal protease. In some embodiments, the methods comprise incubating and/or fermenting sorghum at a temperature conducive to fermentation by the fermentation organism (e.g., 28-38° C.). In some embodiments, the methods comprise incubating the sorghum at a temperature below the starch gelatinization temperature of sorghum in a pretreatment step and then reducing the temperature before addition of the fermenting organism and continuing the process at a temperature of between about 20 and 40° C.

In some aspects, the present invention relates to an enzyme blend or composition comprising a phytase in combination with at least one alpha amylase and glucoamylase, wherein said at least one alpha amylase and/or glucoamylase is a granular starch hydrolyzing enzyme (GSHE) and at least one non-starch polysaccharide hydrolyzing enzyme chosen from cellulases, xylanases, hemicellulases, beta glucanases, beta-glucosidases, and pectinases: The invention also relates to the use of the blend or composition in no-cook processes for fermenting granular sorghum and the production of end-products (e.g., ethanol). In a further aspect the invention relates to an enzyme blend or composition comprising a phytase and at least one GHSE (a GA and/or an AA), and at least one non-starch polysaccharide hydrolyzing enzyme chosen from cellulases, hemicellulases, beta glucanases, xylanases, beta-glucosidases, and pectinases. The GSHE can be an alpha amylase and/or a glucoamylase. In further embodiments, the invention relates to an enzyme blend or composition comprising at least one phytase, at least one alpha amylase with GHSE activity, at least one glucoamylase with GSHE activity and at least two non-starch polysaccharide hydrolyzing enzymes chosen from cellulases, xylanases, hemicellulases, beta glucanases, beta-glucosidases, and pectinases. In a further embodiment, the combination can also comprise at least one acid fungal protease. One advantage of the blend or composition is that it results in a reduced amount of phytic acid in the DDGS. A further advantage of the blend or composition when used during no-cook processes is that it results in increased ethanol production. A further advantage is that it results in the production of nutrients for the yeast involved in fermentation and results in a increased fermentation efficiency.

In some embodiments, the enzyme blend and/or composition is added during the starch hydroysis step and/or the fermentation step of the no-cook process. In some embodiments, the enzyme blend and/or composition is added during a pretreatment step of the no-cook process. In some embodiments, the enzyme blend and/or composition is added during both the pretreatment and the fermentation step of the no-cook process.

In some embodiments, the methods include processes for increasing the fermentation yield of sorghum using at least one phytase together with at least one granular starch hydrolyzing enzyme, and at least one non-starch polysaccharide hydrolyzing enzyme in a no-cook process. The process also includes the addition of a fermentation microorganism simultaneously or separately and incubation of the resulting mixture under suitable fermentation temperatures, but at a temperature below the starch gelatinization temperature of the sorghum to produce ethanol.

In some embodiments, the use of the enzyme(s) in the no-cook process, results in a significant improvement in efficiency of the fermentation, and significant reduction of the phytic acid in the resulting DDGS. A reduction in phytic acid in the DDGS increases the usefulness for feed applications. This is because many feed animals (e.g. non-ruminants like poultry, fish and pigs) are unable to digest the phytic acid. A further disadvantage of phytic acid is that it gets discharged through manure resulting in a phosphate pollution problem.

The invention also relates to the conversion of fermentable sugars from the sorghum to obtain end-products, such as alcohol (e.g., ethanol and butanol), organic acids (lactic acid, citric acid) and specialty biochemical (amino acids, monosodium glutamate, etc).

In some embodiments, the method involves the following steps: 1) contacting granular starch with at least one granular starch hydrolyzing enzyme (AA or GA), at least one phytase and at least one non starch polysaccharide hydrolyzing enzyme at a temperature below the starch gelatinization temperature; 2) reducing the temperature to a temperature between 20° C. and 40° C. and 2) fermenting, wherein the combined time for the incubation and fermentation is between about 10 and 250 hours and wherein the method results in a higher ethanol yield, a higher fermentation efficiency, and/or less phytic acid in the DDGS. Alternatively, secondary enzymes such as proteases can be added.

The at least one phytase, at least one raw starch hydrolyzing enzyme and at least one non-starch polysaccharide hydrolyzing enzyme can be added as a blend or composition or can be added separately during the pretreatment or fermentation steps of the no-cook process. In either case, one advantage of the blend or composition comprising phytase, non-starch polysaccharide hydrolyzing enzymes and GSHEs is that it results in a greater amount of ethanol relative to the amount of ethanol produced by fermentation under substantially the same conditions without the combination of enzymes. In some embodiments, the increase is relative to a method without phytase. In some embodiments, the increase is relative to a method without at least one non-starch polysaccharide hydrolyzing enzyme. In some embodiments, the increase is relative to a method without at least two non-starch polysaccharide hydrolyzing enzymes. In some embodiments, the increase is relative to a method without at least one phytase+at least one non-starch polysaccharide hydrolyzing enzyme. In some embodiments, the increase is relative to the method with the enzymes but using a conventional method rather than a no-cook method. In some aspects, the increase is at least about 0.1%, relative to fermentation without the at least one phytase and non-starch polysaccharide hydrolyzing enzymes, including at least about 0.2%, 0.3%, 0.4%, 0.5%, 0.6%, 0.7%, 0.8%, 0.9%, 1.0%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14% and 15%. In some embodiments, the increase is from about 1% to about 10%, including about 1.1%, 1.2%, 1.3%, 1.4%, 1.5%, 1.6%, 1.7%, 1.8%, 1.9%, 2%, 2.1%, 2.2%, 2.3%, 2.4%, 2.5%, 2.6%, 2.7%, 2.8%, 2.9%, 3%, 3.1%, 3.2%, 3.3%, 3.4%, 3.5%, 3.6%, 3.7%, 3.8%, 3.9%, 4%, 4.1%, 4.2%, 4.3%, 4.4%, 4.5%, 4.6%, 4.7%, 4.8%, 4.9%, 5%, 5.2%, 5.5%, 5.7%, 6%, 6.2%, 6.5%, 6.7%, 7%, 7.2%, 7.5%, 7.7%, 8%, 8.2%, 8.5%, 8.7%, 9%, 9.2%, 9.5%, 9.7%, and 10%. The increase can be relative to any of: 1. a conventional method with or without the enzymes, 2. a method without the addition of the phytase, 3. a method without the addition of the non-starch polysaccharide hydrolyzing enzyme(s), 4. a method without the addition of the non-starch polysaccharide hydrolyzing enzyme(s) and the phytase, and 5. a method without the addition of the non-starch polysaccharide hydrolyzing enzyme(s), the phytase, and the at least one GSHE.

### Phytases—

The specific phytase used in the methods and blends of the invention is not critical to the invention. Phytases are enzymes capable of liberating at least one inorganic phosphate from inositol hexaphosphate. Phytases are grouped according to their preference for a specific position of the phosphate ester group on the phytate molecule at which hydrolysis is initiated, (e.g., as 3-phytases (EC 3.1.3.8) or as 6-phytases (EC 3.1.3.26)). A typical example of phytase is myo-inositol-hexakiphosphate-3-phosphohydrolase. Phytases can be obtained from microorganisms such as fungal and bacterial organisms (e.g. Aspergillus (e.g., A. niger, A. terreus, and A. fumigatus), Myceliophthora (M. thermophila), Talaromyces (T. thermophilus) Trichoderma spp (T. reesei). And Thermomyces (See e.g., WO 99/49740)). Also phytases are available from Penicillium species, (e.g., P. hordei (See e.g., ATCC No. 22053), P. piceum (See e.g., ATCC No. 10519), or P. brevi-compactum (See e.g., ATCC No. 48944) (See, e.g. U.S. Pat. No. 6,475,762). Additional phytases that find use in the invention are available from Peniophora, E. coli, Citrobacter, Enterbacter and Buttiauxella (see e.g., WO2006/043178, filed Oct. 17, 2005). Additional phytases useful in the invention can be obtained commercially (e.g. NATUPHOS® (BASF), RONOZYME® P (Novozymes A/S), PHZYME® (Danisco A/S, Diversa) and FINASE® (AB Enzymes). In some embodiments, the phytase useful in the present invention is one derived from the bacterium Buttiauxiella spp. The Buttiauxiella spp. includes B. agrestis, B. brennerae, B. ferragutiase, B. gaviniae, B. izardii, B. noackiae, and B. warmboldiae. Strains of Buttiauxella species are available from DSMZ, the German National Resource Center for Biological Material (Inhoffenstrabe 7B, 38124 Braunschweig, Germany). Buttiauxella sp. strain P1-29 deposited under accession number NCIMB 41248 is an example of a particularly useful strain from which a phytase can be obtained and used according to the invention. BP-wt and variants such as BP-17 from Buttiauxiella can also be used in the invention (see U.S. patent application Ser. No. 12/027,127, filed Feb. 6, 2008). It is not intended that the present invention be limited to any specific phytase, as any suitable phytase finds use in the methods of the present invention.

### Enzymes Having Granular Starch Hydrolyzing Activity (GSHEs)—

Enzymes having granular starch hydrolyzing activity (GSHEs) are able to hydrolyze granular starch, and these enzymes have been recovered from fungal, bacterial and plant cells such as Bacillus sp., Penicillium sp., Humicola sp., Trichoderma sp. Aspergillus sp. Mucor sp. and Rhizopus sp. In some embodiments, a particular group of enzymes having GSH activity include enzymes having glucoamylase activity and/or alpha-amylase activity (See, Tosi et al., (1993) Can. J. Microbiol. 39:846-855). A Rhizopus oryzae GSHE has been described in Ashikari et al., (1986) Agric. Biol. Chem. 50:957-964 and U.S. Pat. No. 4,863,864. A Humicola grisea GSHE has been described in Allison et al., (1992) Curr. Genet. 21:225-229; WO 05/052148 and European Patent No. 171218. An Aspergillus awamori var. kawachi GSHE has been described by Hayashida et al., (1989) Agric. Biol. Chem. 53:923-929. An Aspergillus shirousami GSHE has been described by Shibuya et al., (1990) Agric. Biol. Chem. 54:1905-1914.

In some embodiments, a GSHE may have glucoamylase activity and is derived from a strain of Humicola grisea, particularly a strain of Humicola grisea var. thermoidea (see, U.S. Pat. No. 4,618,579). In some preferred embodiments, the Humicola enzyme having GSH activity will have at least 85%, 90%, 92%, 94%, 95%, 96%, 97%, 98% and 99% sequence identity to the amino acid sequence of SEQ ID NO: 3 of WO 05/052148.

In other embodiments, a GSHE may have glucoamylase activity and is derived from a strain of Aspergillus awamori, particularly a strain of A. awamori var. kawachi. In some preferred embodiments, the A. awamori var. kawachi enzyme having GSH activity will have at least 85%, 90%, 92%, 94%, 95%, 96%, 97%, 98% and 99% sequence identity to the amino acid sequence of SEQ ID NO: 6 of WO 05/052148.

In other embodiments, a GSHE may have glucoamylase activity and is derived from a strain of Rhizopus, such as R. niveus or R. oryzae. The enzyme derived from the Koji strain R. niveus is sold under the trade name “CU CONC or the enzyme from Rhizopus sold under the trade name GLUZYME.

Another useful GSHE having glucoamylase activity is SPIRIZYME Plus (Novozymes A/S), which also includes acid fungal amylase activity.

In other embodiments, a GSHE may have alpha-amylase activity and is derived from a strain of Aspergillus such as a strain of A. awamori, A. niger, A. oryzae, or A. kawachi and particularly a strain of A. kawachi.

In some preferred embodiments, the A. kawachi enzyme having GSH activity will have at least 85%, 90%, 92%, 94%, 95%, 96%, 97%, 98% and 99% sequence identity to the amino acid sequence of SEQ ID NO: 3 of WO 05/118800 and WO 05/003311.

In some embodiments, the enzyme having GSH activity is a hybrid enzyme, for example one containing a catalytic domain of an alpha-amylase such as a catalytic domain of an Aspergillus niger alpha-amylase, an Aspergillus oryzae alpha-amylase or an Aspergillus kawachi alpha-amylase and a starch binding domain of a different fungal alpha-amylase or glucoamylase, such as an Aspergillus kawachi or a Humicola grisea starch binding domain. In other embodiments, the hybrid enzyme having GSH activity may include a catalytic domain of a glucoamylase, such as a catalytic domain of an Aspergillus sp., a Talaromyces sp., an Althea sp., a Trichoderma sp. or a Rhizopus sp. and a starch binding domain of a different glucoamylase or an alpha-amylase. Some hybrid enzymes having GSH activity are disclosed in WO 05/003311, WO 05/045018; Shibuya et al., (1992) Biosci. Biotech. Biochem 56: 1674-1675 and Cornett et al., (2003) Protein Engineering 16:521-520.

a. Glucoamylases

Various glucoamylases (GA) (E.C. 3.2.1.3.) find use in the present invention as a GSHE and/or a secondary enzyme. In some embodiments, the glucoamylase having use in the invention has granular starch hydrolyzing activity (GSH) or is a variant that has been engineered to have GSH activity. In some embodiments, GSH activity is advantageous because the enzymes act to break down more of the starch in the granular starch in the sorghum or mixed sorghum and/or other grains. In some embodiments, the glucoamylases are endogenously expressed by bacteria, plants, and/or fungi, while in some alternative embodiments, the glucoamylases are heterologous to the host cells (e.g., bacteria, plants and/or fungi). In some embodiments, glucoamylases useful in the invention are produced by several strains of filamentous fungi and yeast. For example, the commercially available glucoamylases produced by strains of Aspergillus and Trichoderma find use in the present invention. Suitable glucoamylases include naturally occurring wild-type glucoamylases as well as variant and genetically engineered mutant glucoamylases (e.g. hybrid glucoamylases). Hybrid glucoamylase include, for example, glucoamylases having a catalytic domain from a GA from one organism (e.g., Talaromyces GA) and a starch binding domain (SBD) from a different organism (e.g.; Trichoderma GA). In some embodiments, the linker is included with the starch binding domain (SBD) or the catalytic domain. The following glucoamylases are nonlimiting examples of glucoamylases that find use in the processes encompassed by the invention. Aspergillus niger G1 and G2 glucoamylase (See e.g., Boel et al., (1984) EMBO J. 3:1097-1102; WO 92/00381, WO 00/04136 and U.S. Pat. No. 6,352,851); Aspergillus awamori glucoamylases (See e.g., WO 84/02921); Aspergillus oryzae glucoamylases (See e.g., Hata et al., (1991) Agric. Biol. Chem. 55:941-949) and Aspergillus shirousami. (See e.g., Chen et al., (1996) Prot. Eng. 9:499-505; Chen et al. (1995) Prot. Eng. 8:575-582; and Chen et al., (1994) Biochem J. 302:275-281).

Additional glucoamylases that find use in the present invention also include those obtained from strains of Talaromyces ((e.g., T. emersonii, T. leycettanus, T. duponti and T. thermophilus glucoamylases (See e.g., WO 99/28488; U.S. Pat. No. RE: 32,153; U.S. Pat. No. 4,587,215)); strains of Trichoderma, (e.g., T. reesei) and glucoamylases having at least about 80%, about 85%, about 90% and about 95% sequence identity to SEQ ID NO: 4 disclosed in US Pat. Pub. No. 2006-0094080; strains of Rhizopus, (e.g., R. niveus and R. oryzae); strains of Mucor and strains of Humicola, ((e.g., H. grisea (See, e.g., Boel et al., (1984) EMBO J. 3:1097-1102; WO 92/00381; WO 00/04136; Chen et al., (1996) Prot. Eng. 9:499-505; Taylor et al., (1978) Carbohydrate Res. 61:301-308; U.S. Pat. No. 4,514,496; U.S. Pat. No. 4,092,434; U.S. Pat. No. 4,618,579; Jensen et al., (1988) Can. J. Microbiol. 34:218-223 and SEQ ID NO: 3 of WO 2005/052148)). In some embodiments, the glucoamylase useful in the invention has at least about 85%, about 90%, about 92%, about 94%, about 95%, about 96%, about 97%, about 98% and about 99% sequence identity to the amino acid sequence of SEQ ID NO: 3 of WO 05/052148. Other glucoamylases useful in the present invention include those obtained from Athelia rolfsii and variants thereof (See e.g., WO 04/111218) and Penicillium spp. (See e.g., Penicillium chrysogenum).

Commercially available glucoamylases useful in the invention include but are not limited to DISTILLASE®, OPTIDEX® L-400 and G ZYME® G990 4X, GC480, G-ZYME 480, FERMGEN® 1-400 (Danisco US, Inc, Genencor Division) CU.CONC® (Shin Nihon Chemicals, Japan), GLUCZYME (Amano Pharmaceuticals, Japan (See e.g. Takahashi et al., (1985) J. Biochem. 98:663-671)). Additional enzymes that find use in the invention include three forms of glucoamylase (E.C.3.2.1.3) produced by a Rhizopus sp., namely “Gluc1” (MW 74,000), “Gluc2” (MW 58,600) and “Gluc3” (MW 61,400). It is not intended that the present invention be limited to any specific glucoamylase as any suitable glucoamylase finds use in the methods of the present invention. Indeed, it is not intended that the present invention be limited to the specifically recited glucoamylases and commercial enzymes.

b. Alpha Amylases

Various alpha amylases find use in the methods of the invention in combination with phytase as a GSHE and/or a secondary enzyme. In some embodiments, the alpha amylase having use in the invention has granular starch hydrolyzing activity (GSH) or is a variant that has been engineered to have GSH activity. In some embodiments, GSH activity is advantageous because the enzymes act to break down more of the starch in the granular starch substrate. Alpha amylases having GSHE activity include, but are not limited to: those obtained from Aspergillus kawachi (e.g., AkAA), Aspergillus niger (e.g., AnAA), and Trichoderma reesei (e.g., TrAA). In some embodiments, the alpha amylase is an acid stable alpha amylase which, when added in an effective amount, has activity in the pH range of 3.0 to 7.0.

Further, in some embodiments, the alpha amylase can be a wild-type alpha amylase, a variant or fragment thereof or a hybrid alpha amylase which is derived from for example a catalytic domain from one microbial source and a starch binding domain from another microbial source. Non-limiting examples of other alpha amylases that can be useful in combination with the blend are those derived from Bacillus, Aspergillus, Trichoderma, Rhizopus, Fusarium, Penicillium, Neurospora and Humicola.

Some of these amylases are commercially available e.g., TERMAMYL® 120-L, LC and SC SAN SUPER®, SUPRA®, and LIQUEZYME® SC available from Novo Nordisk A/S, FUELZYME® FL from Diversa, and CLARASE® L, SPEZYME® FRED, SPEZYME® ETHYL, GC626, and GZYME® G997 available from Danisco, US, Inc., Genencor Division.

It is not intended that the present invention be limited to any specific alpha amylase, as any suitable alpha amylase finds use in the methods of the present invention. Indeed, it is not intended that the present invention be limited to the specifically recited alpha amylase and commercial enzymes.

### Non-Starch Polysaccharide Hydrolyzing Enzymes—

Embodiments of the invention include a composition or blend of at least one phytase, at least one GSHE (an AA and/or a GA), and at least one non-starch polysaccharide hydrolyzing enzyme. Non-starch polysaccharide hydrolyzing enzymes are enzymes capable of hydrolyzing complex carbohydrate polymers such as cellulose, hemicellulose, and pectin. For example, cellulases (endo and exo-glucanases, beta glucosidase) hemicellulases (xylanases) and pectinases are non-starch polysaccharide hydrolyzing enzymes. Thus, in some embodiments, the composition or blend can comprise at least one non-starch polysaccharide hydrolyzing enzyme. In some embodiments, the composition or blend can comprise at least two non-starch polysaccharide hydrolyzing enzymes. In some embodiments, the enzyme composition can comprise at least three non-starch polysaccharide hydrolyzing enzymes chosen from cellulases, xylanases, hemicellulases, beta glucanases, beta-glucosidases, and pectinases. For example, when the blends are used in various applications (e.g. no-cook processing applications) one or more non-starch polysaccharide hydrolyzing enzymes can be included. The blend or composition according to the invention can be used during a pretreatment step and/or during fermentation along with the fermenting microorganism and other components.

Various cellulases find use in the methods according to the invention. Cellulases are enzyme compositions that hydrolyze cellulose (β-1,4-D-glucan linkages) and/or derivatives thereof, such as phosphoric acid swollen cellulose. Cellulases include the classification of exo-cellobiohydrolases (CBH), endoglucanases (EG) and β-glucosidases (BG) (EC3.2.191, EC3.2.1.4 and EC3.2.1.21). Examples of cellulases include cellulases from Penicillium, Trichoderma, Humicola, Fusarium, Thermomonospora, Cellulomonas, Hypocrea, Clostridium, Thermomonospore, Bacillus, Cellulomonas and Aspergillus. Non-limiting examples of commercially available cellulases sold for feed applications are beta-glucanases such as ROVABIO® (Adisseo), NATUGRAIN® (BASF), MULTIFECT® BGL (Danisco Genencor) and ECONASE® (AB Enzymes). Some commercial cellulases includes ACCELERASE®. The cellulases and endoglucanases described in US20060193897A1 also may be used. Beta-glucosidases (cellobiase) hydrolyzes cellobiose into individual monosaccharides. Various beta glucanases find use in the invention in combination with phytases. Beta glucanases (endo-cellulase-enzyme classification EC 3.2.1.4) also called endoglucanase I, II, and III, are enzymes that will attack the cellulose fiber to liberate smaller fragments of cellulose which is further attacked by exo-cellulase to liberate glucose. □-glucanases can also be used in the methods according to the invention. Commercial beta-glucanases useful in the methods of the invention include OPTIMASH® BG and OPTIMASH® TBG (Danisco, US, Inc. Genencor Division). It is not intended that the present invention be limited to any specific beta-glucanase, as any suitable beta-glucanase finds use in the methods of the present invention.

Numerous cellulases have been described in the scientific literature, examples of which include: from Trichoderma reesei: Shoemaker, S. et al., Bio/Technology, 1:691-696, 1983, which discloses CBHI; Teen, T. et al., Gene, 51:43-52, 1987, which discloses CBHII; Penttila, M. et al., Gene, 45:253-263, 1986, which discloses EGI; Saloheimo, M. et al., Gene, 63:11-22, 1988, which discloses EGII; Okada, M. et al., Appl. Environ. Microbiol., 64:555-563, 1988, which discloses EGIII; Saloheimo, M. et al., Eur. J. Biochem., 249:584-591, 1997, which discloses EGIV; Saloheimo, A. et al., Molecular Microbiology, 13:219-228, 1994, which discloses EGV; Barnett, C. C., et al., Bio/Technology, 9:562-567, 1991, which discloses BGLJ, and Takashima, S. et al., J. Biochem., 125:728-736, 1999, which discloses BGL2. Cellulases from species other than Trichoderma have also been described e.g., Ooi et al., 1990, which discloses the cDNA sequence coding for endoglucanase F1-CMC produced by Aspergillus aculeatus; Kawaguchi T et al., 1996, which discloses the cloning and sequencing of the cDNA encoding beta-glucosidase 1 from Aspergillus aculeatus; Sakamoto et al., 1995, which discloses the cDNA sequence encoding the endoglucanase CMCase-1 from Aspergillus kawachii IFO 4308; Saarilahti et al., 1990 which discloses an endoglucanase from Erwinia carotovara; Spilliaert R, et al., 1994, which discloses the cloning and sequencing of bglA, coding for a thermostable beta-glucanase from Rhodothermus marinu; and Halldorsdottir S et al., 1998, which discloses the cloning, sequencing and overexpression of a Rhodothermus marinus gene encoding a thermostable cellulase of glycosyl hydrolase family 12. It is not intended that the present invention be limited to any specific cellulase, as any suitable cellulase finds use in the methods of the present invention. Indeed, it is not intended that the present invention be limited to the specifically recited cellulases and commercial enzymes.

Hemicellulases are enzymes that break down hemicellulose. Hemicellulose categorizes a wide variety of polysaccharides that are more complex than sugars and less complex than cellulose, that are found in plant walls. In some embodiments, a xylanase find use as a secondary enzyme in the methods of the invention. Any suitable xylanase can be used in the invention. Xylanases (e.g. endo-β-xylanases (E.C. 3.2.1.8), which hydrolyze the xylan backbone chain, can be from bacterial sources (e.g., Bacillus, Streptomyces, Clostridium, Acidothermus, Microtetrapsora or Thermonospora) or from fungal sources (Aspergillus, Trichoderma, Neurospora, Humicola, Penicillium or Fusarium (See, e.g., EP473 545; U.S. Pat. No. 5,612,055; WO 92/06209; and WO 97/20920)). Xylanases useful in the invention include commercial preparations (e.g., MULTIFECT® and FEEDTREAT® Y5 (Danisco Genencor), RONOZYME® WX (Novozymes A/S) and NATUGRAIN WHEAT® (BASF). In some embodiments the xylanase is from Trichoderma reesei or a variant xylanase from Trichoderma reesei, or the inherently thermostable xylanase described in EP1222256B1, as well as other xylanases from Aspergillus niger, Aspergillus kawachii, Aspergillus tubigensis, Bacillus circulans, Bacillus pumilus, Bacillus subtilis, Neocallimastix patriciarum, Penicillium species, Streptomyces lividans, Streptomyces thermoviolaceus, Thermomonospora fusca, Trichoderma harzianum, Trichoderma reesei, Trichoderma viridae.

### Secondary Enzymes—

Secondary enzymes include without limitation: additional glucoamylases, additional alpha amylases additional cellulases, additional hemicellulases, xylanases, additional proteases, phytases, pullulanases, beta amylases, lipases, cutinases, additional pectinases, additional beta-glucanases, galactosidases, esterases, cyclodextrin transglycosyltransferases (CGTases), alpha galactosidases, dextrinases, beta-amylases and combinations thereof. Any additional alpha amylases, glucoamylases, proteases, cellulases, pectinases, beta glucanases, and phytases that are known or are developed can be used, including those disclosed herein.

Various acid fungal proteases (AFP) find use in the methods of the invention. Acid fungal proteases include for example, those obtained from Aspergillus, Trichoderma, Mucor and Rhizopus, such as A. niger, A. awamori, A. oryzae and M. miehei. AFP can be derived from heterologous or endogenous protein expression of bacteria, plants and fungi sources. In particular, AFP secreted from strains of Trichoderma find use in the invention. Suitable AFP includes naturally occurring wild-type AFP as well as variant and genetically engineered mutant AFP. Some commercial AFP enzymes useful in the invention include FERMGEN® (Danisco US, Inc, Genencor Division), and FORMASE® 200.

In some embodiments, the acid fungal protease useful in the invention will have at least about 85%, 90%, 92%, 94%, 95%, 96%, 97%, 98% and 99% sequence identity to the amino acid sequence of SEQ ID NO:14 (see U.S. patent application Ser. No. 11/312,290, filed Dec. 20, 2005). It is not intended that the present invention be limited to any specific acid fungal protease, as any suitable acid fungal protease finds use in the methods of the present invention. Indeed, it is not intended that the present invention be limited to the specifically recited acid fungal protease and commercial enzymes.

Additional proteases can also be used with the blends and/or compositions according to the invention other than AFPs. Any suitable protease can be used. Proteases can be derived from bacterial or fungal sources. Sources of bacterial proteases include proteases from Bacillus (e.g., B. amyloliquefaciens, B. lentus, B. licheniformis, and B. subtilis). Exemplary proteases include, but are not limited to, subtilisin such as a subtilisin obtainable from B. amyloliquefaciens and mutants thereof (U.S. Pat. No. 4,760,025). Suitable commercial protease includes MULTIFECT® P 3000 (Danisco Genencor) and SUMIZYME® FP (Shin Nihon). Sources of suitable fungal proteases include, but are not limited to, Trichoderma, Aspergillus, Humicola and Penicillium, for example.

### Blends/Compositions—

The blends and compositions of the invention include at least one phytase in combination with an alpha amylase, a glucoamylase (wherein at least one of the alpha amylase and/or glucoamylase is a GHSE), and at least one non-starch polysaccharide hydrolyzing enzyme. In some embodiments, both the alpha amylase and glucoamylase is a granular starch hydrolyzing enzyme. The non-starch polysaccharide hydrolyzing enzyme can be chosen from a cellulase, a hemicellulases, a beta glucosidase, and a pectinase. In some embodiments, the blends and or composition used in no-cook application comprise at least one phytase, at least one alpha amylase (AA), at least one glucoamylase (GA), at least one cellulase, and at least one acid fungal protease. In some embodiments, the blends and/or compositions include at least one phytase, at least one alpha amylase (AA), at least one glucoamylase (GA), at least one cellulase, at least one pectinase, at least one beta glucanase, at least one beta-glucosidase, and at least one acid fungal protease (AFP). The enzyme components can be used as a blended formulation comprising two or more enzyme components mixed together or the enzyme components can be individually added during a process step to result in a composition encompassed by the invention. The compositions of the invention can be used during a step in the fermentation such that a formulation is maintained. This may involve adding the separate components of the composition in a time-wise manner such that the formulation is maintained, for example adding the components simultaneously.

The phytase can be provided in an amount effective to reduce the phytic acid in the DDGS and/or the thin stillage. In some embodiments, the phytase is added in an amount effective to increase the amount of inositol and/or phosphate. In some embodiments, the amount of phytase is at least 0.01 FTU/g DS, including at least 0.02, 0.03, 0.04, 0.05, 0.06, 0.07, 0.08, 0.09, 0.10, 0.11, 0.12, 0.13, 0.14, 0.15, 0.16, 0.17, 0.18, 1.9, 2.0, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, and 100 FTU/g DS. In some embodiments, phytase is added in an amount from about 0.01 FTU/g DS to about 100 FTU/g DS or more. In some embodiments, the phytase is added from about 2.0 to about 50 FTU/g DS. In some embodiments, the phytase is added from about 1 to about 10 FTU/g DS.

The blends and compositions of the invention include at least one phytase. In some embodiments, the phytase is used in combination with at least one AA, at least one GA (wherein the at least one AA and/or at least one GA has granular starch hydrolyzing activity) and at least one non-starch polysaccharide hydrolyzing enzyme. In other embodiments, the granular starch hydrolyzing enzyme is a glucoamylase and an alpha amylase. In other embodiments, the blends or compositions of the invention include at least one phytase, at least one alpha amylase with GSH activity, at least one glucoamylase with GSHE, at least one cellulase and at least one other non-starch polysaccharide hydrolyzing enzyme.

A composition comprising a GHSE glucoamylase and a GSHE alpha amylase, which is useful in combination with the phytase is STARGEN™ 001, which is a blend of an acid stable alpha amylase and a glucoamylase (available commercially from Danisco US, Inc., Genencor Division). To this can be added the other enzymes as disclosed herein.

In some embodiments, the GSHE is an alpha amylase and the effective dose in the contacting step and/or fermentation step will be 0.01 to 15 SSU/g DS; also 0.05 to 10 SSU/g DS; also 0.1 to 10 SSU/g DS; and 0.5 to 5 SSU/g DS.

In some embodiments, the effective dose of a glucoamylase for the contacting step and/or the fermentation step will be in the range of 0.01 to 15 GAU/g DS; also 0.05 to 10 GAU/g DS; also 0.1 to 10 GAU/g DS and even 0.5 to 5 GAU/g DS.

### E. Sorghum—

Agronomically, sorghum is a common name applied to plants in the genus Sorghum. The cultivars of particular interest are the grain sorghums. Sorghum is also referred to in various parts of the world as millet and also milo.

Most industrial plants using conventional processes for producing ethanol from milled corn average 92% fermentation efficiency. The fermentation efficiency for sorghum is much lower. When evaluating sorghum as a fermentation feedstock for ethanol production, a number of factors may affect fermentation yield and digestibility. Reference is made to Table 1, wherein some of the factors are listed. The conventional process for producing soluble dextrins from insoluble starch involves heating the whole ground grain or starch slurry to greater than 95° C. in the presence of thermostable alpha amylase for liquefaction followed by cooling, pH adjustment and subsequent fermentation in the presence of glucoamylase and yeast for conversion to ethanol. However, a lower fermentation efficiency resulted in a lower alcohol yield for sorghum as compared with corn using this process (See, e.g., Enzeogen, et al. 2005, J. Cereal Sci. 42:33-44; and Duodu et. al; 2004, J. Cereal Sci. 38:117-131). Sorghum is also known to be less digestible in animals as compared with corn, especially after sorghum has been exposed to elevated temperatures that are encountered during high temperature/pressure jet-cooking (See, e.g., Duodu et al 2004 supra).

TABLE 1
Factors Effecting Fermentation Yield and Digestibility

| Parameter | Effect |
|---|---|
| Tannin | Lined with pigment testa. Liquefaction of starch containing tannin in |
| | sorghum is more difficult and slower due to higher viscosity. (See, e.g., Wu |
| | et al 2007, 84.131-136). Tannin also complexes with enzymes resulting in |
| | reduced enzyme activity affecting starch and protein digestibility |
| Non-starch | 2-7% NSP, (Arabinoxylans-35% and Glucan-40%). Forms viscous and |
| polysaccharides | sticky solutions resulting in poor separation. |
| (NSP) | |
| Phytic acid | 1.2-1.8% phytic acid. Phytic acid in sorghum impacts the ethanol process |
| | economics resulting in: 1) Phosphate disposal/environmental pollution 2) |
| | Binding of trace metals and decreased digestibility of proteins by proteolytic |
| | enzymes impacting the yeast growth. It also results in lower starch |
| | hydrolysis because of alpha amylase inhibitory effect. |
| | (See, e.g., Shetty, J; et al. (2007) Paper Presented at 2007 Fuel Ethanol |
| | Workshop and Expo, St. Louis, MO, June 26-29) |
| Proteins | 7-15% proteins. Protein digestibility decreases with cooking. (See, e.g., |
| | Duodu, et al. 2004 supra) |
| Bran | Removal of bran by decortications reduces tannin and other |
| | fermentation inhibitors-phenolic acids, color compounds and |
| | improves protein digestibility. |
| Disulphide or | Formation of web-like protein structures during cooking of sorghum |
| Protein cross-linking | results in small starch granules highly trapped within the web-like |
| | protein matrices makes starch unavailable for enzymatic hydrolysis (See, |
| | e.g., Hamker, B et al. .2004 Over view: Sorghum proteins and Food |
| | quality.In:Proc.AFRIPRO Workshop on the Proteins of Sorghum and |
| | Millets: Enhancing Nutritional and Functional Properties for Africa. P. S. Belton |
| | and J. R. N. Taylor, eds. Available at the website for afripro.org.uk |
| | Pretoria, South Africa.) |
| Reduction in starch | Cooked sorghum had lower starch digestibility (15-25%) compared |
| digestibility of | to maize (See, e.g., Zhang, G; et al.1998, Cereal Chem.75: 710-713) |
| cooked sorghum | |
| starch by AA | |
| Amylose-lipid | Decrease the digestibility of starch (See e.g., Wu. X; Zhao, R; Bean, S. R; |
| complexes | Seib, P. A; McLaren, J. S; Madl, R. L.; Tuinstra, M.; Lenz, M. C; and |
| | Wang. D. (2007) Cereal Chem. 84: 130-136) |
| B-complex vitamins | Essential for yeast but destroyed during the high temperature jet-cooking |
| (thiamin, riboflavin | process. |
| and niacin, etc) | |

The phytic acid content (mg/g) of different commercial flours can be compared (see Table 2 adapted from “Phytic acid content in milled cereal products and breads”, Carcia-Estepa, et. al 1999, Food Research Intl 32: 217-221).

TABLE 2
Phytic acid content of commercial flours

| | Amount (mg of phytic |
|---|---|
| Flours | acid/g of grain) |
| Barley | 6.32 |
| Corn | 10.78 |
| Millet | 10.64 |
| Oat | 7.44 |
| Rice | 5.52 |
| Rye | 4.52 |
| Sorghum | 10.12 |
| Wheat | 4.04 |
| Whole wheat | 22.20 |

Table 2 shows that corn, millet, and sorghum flours contained approximately 10 mg/g of phytic acid. The values of phytic acid are typically higher in the bran than in the endosperm of the grains. Some grains contain naturally occurring phytase enzymes that could potentially be used to remove at least some of the phytic acid. These include Rye, Wheat bran, Wheat, and Barley. However, Corn, Sorghum and rice contain less than 20 phytase units/Kg (See, e.g., Ravindran, V.; Bryden, W. L.; Kornegay, E. T. 1995. Phytates: occurrence, bioavailability and implications in poultry nutrition. Poultry and Avian Biology Reviews, 6(2), 125-143). Thus, sorghum contains high amounts of phytic acid and very little phytase activity to digest the phytic acid.

### F. Methods of Use

In some embodiments, the sorghum to be processed is mixed with an aqueous solution to obtain a slurry. The aqueous solution can be obtained, for example from water, thin stillage and/or backset. In some embodiments, the slurry has a DS of between 5-60%; 10-50%; 15-45%; 15-30%; 20-45%; 20-30% and also 25-40%.

In some embodiments, the slurry is contacted with the enzyme blend or composition during the fermentation. In some embodiments, the slurry is contacted with the enzyme blend or composition during a pretreatment and before fermentation. In some embodiments, the enzyme blend and/or composition is added both during a pretreatment and during fermentation. The slurry can be contacted with the at least one phytase, at least one GSHE, at least one non-starch polysaccharide hydrolyzing enzyme and/or enzyme blend or composition of the invention in a single dose or a split dose as long as the formulation of enzymes is maintained. Thus, a split dose means that the total dose in the desired formulation is added in more than one portion, including two portions or three portions. In some embodiments, one portion of the total dose is added at the beginning and a second portion is added at a specified time in the process. In some embodiments, at least a portion of the dose is added as a pretreatment. In some embodiments, at least one of the enzymes in the enzyme blend or composition of the invention can be immobilized on a column or solid substrate.

The enzyme blend or composition can be added at a temperature below the gelatinization temperature of the granular starch in the sorghum during a pretreatment and/or fermentation step. In some embodiments, the enzyme blend and/or composition is added at a temperature conducive to fermentation by the fermenting organism, such as at 20-40° C. during the fermentation step. Alternatively, the pretreatment can be conducted at a temperature below the starch gelatinization temperature of the sorghum. In some embodiments, this temperature is between 20° C. and 90° C.; in other embodiments, the temperature is held between 50° C. and 77° C.; between 55° C. and 77° C.; between 60° C. and 70° C., between 60° C. and 65° C.; between 55° C. and 65° C. and between 55° C. and 68° C. In further embodiments, the temperature is at least 45° C., 48° C., 50° C., 53° C., 55° C., 58° C., 60° C., 63° C., 65° C. and 68° C. In other embodiments, the temperature is not greater than 65° C., 68° C., 70° C., 73° C., 75° C. and 80° C.

In some embodiments, if the pretreatment is conducted at a temperature less than the gelatinization temperature of sorghum, but above the fermentation temperature of the fermenting organism, the temperature is reduced before addition of the fermenting organism.

The pretreatment and/or fermentation can be conducted at a pH ranging from pH 3.5 to 7.0; also at a pH range of 3.5 to 6.5; also at a pH range of 4.0 to 6.0 and in some embodiments at a pH range of 4.2 to 5.5. In some embodiments, the pretreatment is conducted at a pH closest to the pH optimum of one or more of the enzymes in the enzyme blend and/or composition.

In some embodiments the pretreated molasses is subjected to fermentation with fermenting microorganisms. In some embodiments, the contacting step (pretreatment) and the fermenting step can be performed simultaneously in the same reaction vessel or sequentially. In general, fermentation processes are described in The Alcohol Textbook 3^{rd} ED, A Reference for the Beverage, Fuel and Industrial Alcohol Industries, Eds Jacques et al., (1999) Nottingham University Press, UK.

The slurry can be held in contact with the enzyme blend and or composition during a pretreatment and/or fermentation step for a period of 5 minutes to 120 hours; and also for a period of 5 minutes to 66 hours, 5 minutes to 24 hours. In some embodiments the period of time is between 15 minutes and 12 hours, 15 minutes and 6 hours, 15 minutes and 4 hours and also 15 minutes and 2 hours. In some embodiment, if there is a pretreatment step the combination of pretreatment and fermentation is conducted for a period of 5 minutes to 120 hours, including any of the above ranges.

In some embodiments the slurry is subjected to fermentation with fermenting microorganisms. In some embodiments, the fermenting organism is a yeast. During fermentation, the fermentable sugars (dextrins e.g. glucose) in the sorghum are used in microbial fermentations under suitable fermentation conditions to obtain end-products, such as alcohol (e.g., ethanol), organic acids (e.g., succinic acid, lactic acid), sugar alcohols (e.g., glycerol), ascorbic acid intermediates (e.g., gluconate, DKG, KLG), and amino acids (e.g., lysine).

In some embodiments, the fermentable sugars are fermented with a yeast at temperatures in the range of 15 to 40° C., 20 to 38° C., and also 25 to 35° C.; at a pH range of pH 3.0 to 6.5; also pH 3.0 to 6.0; pH 3.0 to 5.5, pH 3.5 to 5.0 and also pH 3.5 to 4.5 for a period of time of 5 hrs to 120 hours, preferably 12 to 120 and more preferably from 24 to 90 hours to produce an alcohol product, preferably ethanol.

Yeast cells are generally supplied in amounts of 10⁴ to 10¹², and preferably from 10⁷ to 10¹⁰ viable yeast count per ml of fermentation broth. The fermentation will include in addition to a fermenting microorganism (e.g. yeast) nutrients, optionally acid and enzymes. In some embodiments, in addition to the raw materials described above, fermentation media will contain supplements including but not limited to vitamins (e.g. biotin, folic acid, nicotinic acid, riboflavin), cofactors, and macro and micro-nutrients and salts (e.g. (NH4)₂SO₄; K₂HPO₄; NaCl; MgSO₄; H₃BO₃; ZnCl₂; and CaCl₂).

In some embodiments, in addition to the raw materials described above, fermentation media will contain supplements including but not limited to vitamins (e.g. biotin, folic acid, nicotinic acid, riboflavin), cofactors, and macro and micro-nutrients and salts (e.g. (NH₄)₂SO₄; K₂HPO₄; NaCl; MgSO₄; H₃BO₃; ZnCl₂; and CaCl₂).

### G. Recovery of Alcohol, DDGS and Other End-Products—

In some embodiments, an end-product of the instant fermentation process is an alcohol product, (e.g. ethanol or butanol). In some embodiments, the end-product produced according to methods of the invention can be separated and/or purified from the fermentation media. Methods for separation and purification are known in the art and include methods such as subjecting the media to extraction, distillation and column chromatography. In some embodiments, the end-product is identified directly by submitting the media to high-pressure liquid chromatography (HPLC) analysis.

In further embodiments, end-products such as alcohol and solids can be recovered by centrifugation. In some embodiments, the alcohol is recovered by means such as distillation and molecular sieve dehydration or ultra filtration. In some embodiments, the ethanol is used for fuel, portable or industrial ethanol.

In further embodiments, the end-product can include the fermentation co-products such as distillers dried grains (DDG) and distiller's dried grain plus solubles (DDGS), which can be used as an animal feed. In some embodiments, the enzyme composition can reduce the phytic acid content of the fermentation broth, the phytate content of the thin stillage and/or the phytic acid content of co-products of the fermentation such as Distillers Dried Grains (DDG); Distillers Dried Grains with Solubles (DDGS); Distillers wet grains (DWG) and Distillers wet grains with solubles (DWGS). In some embodiments, the methods of the invention (including but not limited to, for example, incubation for 30 to 60 minutes) can reduce the phytic acid content of the resulting fermentation filtrate by at least about 60%, at least about 65%, at least about 70%, at least about 75%, at least about 80%, at least about 85% and at least about 90% and greater as compared to essentially the same process without the phytase. In some embodiments, the amount of phytate found in the DDGS can be reduced by at least about 50%, at least about 70%, at least about 80% and at least about 90% as compared to the phytate content in DDGS from a corresponding process which is essentially the same as the claimed process but without a phytase pretreatment incubation according to the invention. For example, while the % phytate content in commercial samples of DDGS can vary, a general range of % phytate can be from about 1% to about 3% or higher. In some embodiments, the % phytate in the DDGS obtained from the current process will be less than about 1.0%, less than about 0.8% and also less than about 0.5%. In some embodiments the DDGS can be added to an animal feed before or after pelletization. In some embodiments, the DDGS can include an active phytase. In some embodiment the DDGS with the active phytase can be added to an animal feed.

In some industrial ethanol processes, ethanol is distilled from the filtrate resulting in a thin stillage portion that is suitable for recycling into the fermentation stream. The present invention results in thin stillage from similar methods, but that have a lower phytic acid content as compared to the phytate content of thin stillage from a corresponding process which is essentially the same as the claimed process. In some embodiments, the reduction in phytic acid is due to phytase pretreatment incubation step. In some embodiments, the phytase is added during saccharification and/or saccharification/fermentation steps. In some embodiments, methods of the invention (including but not limited to, for example, incubation of 30 to 60 minutes as a pretreatment or during SSF) can reduce the phytic acid content of the resulting thin stillage by at least about 60%, 65%, 70%, 75%, 80%, 85% and 90% and greater as compared to essentially the same process without the phytase. In some embodiments, the amount of phytate found in the thin stillage can be reduced by at least about 50%, at least about 60%, at least about 70%, at least about 80% and at least about 90% as compared to the phytate content in thin stillage from a corresponding process which is essentially the same as the claimed process but without a phytase treatment incubation according to the invention.

In further embodiments, by use of appropriate fermenting microorganisms as known in the art, the fermentation end-product can include without limitation ethanol, glycerol, 1,3-propanediol, gluconate, 2-keto-D-gluconate, 2,5-diketo-D-gluconate, 2-keto-L-gulonic acid, succinic acid, lactic acid, amino acids and derivatives thereof. More specifically when lactic acid is the desired end-product, a Lactobacillus sp. (L. casei) can be used; when glycerol or 1,3-propanediol are the desired end-products E. coli can be used; and when 2-keto-D-gluconate, 2,5-diketo-D-gluconate, and 2-keto-L-gulonic acid are the desired end-products, Pantoea citrea can be used as the fermenting microorganism. The above enumerated list are only examples and one skilled in the art will be aware of a number of fermenting microorganisms that can be appropriately used to obtain a desired end-product.

### EXPERIMENTAL

The present invention is described in further detail in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

In the disclosure and experimental section which follows, the following abbreviations apply: % w/w (weight percent); ° C. (degrees Centigrade); H₂O (water); dH₂O (deionized water); dIH₂O (deionized water, Milli-Q filtration); g or gm (grams); μg (micrograms); mg (milligrams); kg (kilograms); μl (microliters); mL and ml (milliliters); mm (millimeters); μm (micrometer); M (molar); mM (millimolar); μM (micromolar); U (units); MW (molecular weight); sec (seconds); min(s) (minute/minutes); hr(s) (hour/hours); DO (dissolved oxygen); W/V (weight to volume); W/W (weight to weight); V/V (volume to volume); Genencor (Danisco US Inc, Genencor Division, Palo Alto, Calif.); Ncm (Newton centimeter), ETOH (ethanol). Eq (equivalents); N (Normal); ds or DS (dry solids content) MT (metric ton).

In the following examples the materials and methods used were:

Starch Content Determination of Whole Grains Grains were mixed with MOPS buffer (50 mM, pH 7.0) plus calcium chloride (5 mM) and the pH adjusted with Acetic Acid Solution (2N) Sodium hydroxide (2N); Acetate Buffer (pH 4.2) was prepared as follows: 200 ml of 2N acetic acid to 500 ml of water. Using a standardized pH meter, add 2N sodium hydroxide to the mixture until the buffer is 4.2+/−0.05. SPEZYME® FRED (Genencor International alpha-amylase from Bacillus licheniformis) and OPTIDEX® L-400 (Genencor International glucoamylase from Aspergillus niger) are added and the starch content determined by HPLC.

Carbohydrate and Alcohol Analysis by High Pressure Liquid Chromatographic (HPLC): The composition of the reaction products of oligosaccharides was measured by HPLC (Beckman System Gold 32 Karat Fullerton, Calif. equipped with a HPLC column (Rezex 8 u8% H, Monosaccharides), maintained at 50° C. fitted with a refractive index (RI) detector (ERC-7515A RI Detector, Anspec Company Inc.). Saccharides were separated based on molecular weight. A designation of DP1 is a monosaccharide, such as glucose; a designation of DP2 is a disaccharide, such as maltose; a designation of DP3 is a trisaccharide, such as maltotriose and the designation “DP4⁺” is an oligosaccharide having a degree of polymerization (DP) of 4 or greater.

Alpha amylase activity (AAU) can be determined by the rate of starch hydrolysis, as reflected in the rate of decrease of iodine-staining capacity measured spectrophotometrically. One AAU of bacterial alpha-amylase activity is the amount of enzyme required to hydrolyze 10 mg of starch per min under standardized conditions.

Alpha-amylase activity can also be determined as soluble starch unit (SSU) and is based on the degree of hydrolysis of soluble potato starch substrate (4% DS) by an aliquot of the enzyme sample at pH 4.5, 50° C. The reducing sugar content is measured using the DNS method as described in Miller, G. L. (1959) Anal. Chem. 31:426-428.

Glucoamylase Activity Units (GAU) is determined by using the PNPG assay to measure the activity of glucoamylase. GAU is defined as the amount of enzyme that will produce 1 g of reducing sugar calculated as glucose per hour from a soluble starch substrate at pH 4.2 and 60° C.

Fermentation efficiency is the percent actual weight of ethanol produced compared to the theoretical weight of ethanol from a glucose producing substrate i.e. actual starch using the following formula as described (Yeast to Ethanol, 1993, 5, 2^{nd} edition, 241-287, Academic Press, Ltd.). The total starch content on a dry weight basis, conversion of starch to fermentable sugars by enzymatic hydrolysis during fermentation and chemical grain from starch to glucose is taken into consideration.

For example, one ton of sorghum at 12% moisture contains 880 Kg of dry sorghum. The starch content of a particular weight of sorghum is 64.5% (dry weight) or 567.6 Kg of starch. The complete hydrolysis of 567.6 Kg. of dry starch results in 624.36 Kg of glucose (11% chemical grain due to hydrolysis). The theoretical yield of alcohol from glucose is 52.1%, therefore yielding 318.42 Kg of ethanol, or 404.66 liters. It has been reported that the fermentation efficiency for sorghum using a conventional no-cook process is generally in between 86 to 88%. ( ).

$$\% Fermentation Efficiency = \frac{Weight of ethanol produced \times 100}{Theoretical weight of ethanol from produced glucose}$$

Phytase Activity (FTU) was measured by the release of inorganic phosphate. The inorganic phosphate forms a yellow complex with acidic molybdate/vandate reagent and the yellow complex was measured at a wavelength of 415 nm in a spectrophometer and the released inorganic phosphate was quantified with a phosphate standard curve. One unit of phytase (FTU) is the amount of enzyme that releases 1 micromole of inorganic phosphate from phytate per minute under the reaction conditions given in the European Standard (CEN/TC 327,2005-TC327WI 003270XX).

Nitric Thorium method of determining phytic acid content—The method uses the fact that phytate and thorium ions chelate at ratio of 1:2 in a pH=1.6˜3.5 solution. The phytic acid was titrated with standard nitric thorium and excess thorium ions were determined by a color change upon addition of the indicator xylenol orange (pink). The reagents used were 0.02 mol/L Standard Nitric Thorium solution (Nitric Thorium: AR, from Beijing lanthanum innovation company), 0.02 mol/L Standard EDTA-2Na solution, and 0.1% xylenol orange indicator. The procedure was as follows: 1. The solution was calibrated with nitric thorium in a 0.02 mol/L Standard EDTA-2Na solution. Then 0.100 g of sample (a higher amount was used if the phytic acid content in the sample was low) was dissolved in 30˜50 ml of purified water and the pH was adjusted to a pH=1.9˜2.2 with 0.2 mol/L HNO₃. The solution containing the sample was heated to 60° C. and 2˜3 drops of 0.1% xylenol orange were added. The solution was titrated with nitric thorium quickly and the endpoint determined by a color changed from yellow to pink that did not disappear within 30 s. The phytate content was determined as follows:

$$Phytate Content = \frac{M V \times 660 \times 1 \text{/} 2}{1000 m} \times 100 \%$$

$$M \text{:} Concentration of Standard Nitric Thorium solution , mol \text{/} L$$

$$V \text{:} Titration volume of Standard Nitric Thorium solution , ml$$

$$m \text{:} Sample weight , g$$

$$660 \text{:} molar mass of phytate , g \text{/} mol$$

$$1 \text{/} 2 \text{:} chelating ratio of Phytate and Nitric Thorium$$

### Materials—

Thus, in some embodiments, the present invention discloses a formulation composed of phytase and other enzymes such as those discussed above which can be used to improve the yield of ethanol in a fermentation of sorghum in no-cook processes and to reduce the amount of phytic acid in the DDGS produced from the process.

Materials—enzymes—The following enzymes were used in the examples: Buttiauxiella phytase (BP-17), STARGEN 004, STARGEN 001, All were obtained from Danisco US, Inc. Genencor Division.

Experimental—The fermentations were carried out as explained in example 1 with different DS, particle size. In a typical experiment, sorghum with or without hull were selected 100% by passing through a 30 mesh.

The moisture content of these grains was measured using a SARTORIUS AG GOTTINGEN MA 30-000V3 balance (Germany). In each flask, 55-60 grams (based on the moisture content) of the raw material and 145 or 140 grams of tap water were taken and 400 ppm Urea (based on DS) was then added. The pH of the slurry was adjusted to pH 4.2 using 26% sulphuric acid. STARGEN 001 (Genencor, Danisco, USA) was added at 0.7 GAU/g.ds based on the ds. The flask was then inoculated with 0.4% (based on DS) dry Angel yeast (Hubei Angel Yeast Co., Ltd). The fermentation medium was constantly mixed with a slow agitation in a 30° C. water bath. The fermentations were terminated at 66˜67 hours, 2 ml of fermentation broth supernatant was analyzed by HPLC and distillation was carried out with 100 ml of whole broth, the residual starch content was determined using the fermentor broth sample from about 66 to ˜67 hours.

HPLC method for fermentation broth analysis—An Agilent 1100, Column specification: BIO-RAD Aminex HPX-87H or Rezex RoA-organic acid. Method of analysis: ESTD. Details of the analysis: Mobile phase: 0.005 mol/L H₂SO₄. Sample was withdrawn and diluted 10 times, and Filtered using 0.45 nm filter membrane. Other details of the HPLC: Injection volume: 20 μL; Pump flow: 0.6 ml/min; Column thermostat temperature: 60° C.; RID, optical unit temperature: 35° C. Analysis method: ESTD.

Phytic acid amount was determined using the nitric thorium assay above.

### Example 1

### Effect of Phytase on Red Sorghum without Hull

Red sorghum from a local supermarket in Wuxi China (Wuxi Darunfa, China), de-hulled (also called white sorghum) was ground using a FOSS 1093 miller, and then screened 100% by passing through a mesh screen to produce 30 mesh powders. 142.8 g water was added to 57.2 g of the sorghum powder to produce a slurry. Yeast was added at 0.4% of the dry weight. Urea at 400 ppm was also added to a pH of about 5.0 or less. BP-17 phytase was added at 2.2 FTU/g DS, 8.8 FTU/g DS, or 22 FTU/g DS. The three doses of phytase were added as shown in Table 3. The control contained no phytase. STARGEN 001 (Alpha amylase (AA) and glucoamylase (GA)) were also added at 2000 SSU AA and 400 GAU GA. Fermentations were conducted in a 500 ml Erlenmeyer flask and incubated a 30° C. bath with an agitation speed of 150 rpm. The fermentations were terminated at 66 hours and samples of the fermentation broth were taken for HPLC analysis. Distillation of the fermentation whole broth was carried out for calculating the ethanol yield per metric ton of sorghum. The results of the fermentation are shown in Table 3. In the Table the ethanol yield is given with respect to 1 MT sorghum to 95.5% ethanol (L) at 20° C. When using conventional methods to distill ethanol, 95.5% is the maximum amount that can be achieved at 20° C. The abbreviations used in the Table are as follows: Gluc (Glucose); Fruc (Fructose); Suc acid (Succinic acid); Lac acid (Lactic acid); Glyc (Glycerol); EtOH (ethanol).

TABLE 3
Effect of Phytase on de-hulled red sorghum from Wuxi, China:

| | | | | | | % | % | | | |
|---|---|---|---|---|---|---|---|---|---|---|
| | % | % | % | | % | w/v | w/v | % | | EtOH |
| Sample | w/v | w/v | w/v | % w/v | w/v | suc | lact | w/v | % v/v | yield/ |
| name | DP > 3 | DP-3 | DP-2 | Gluc | Fruc | acid | acid | glyc | EtOH | MT |
| Control | 0.11 | 0.00 | 0.12 | 0.02 | 0.00 | 0.13 | 0.06 | 0.84 | 14.86 | 436 |
| without | | | | | | | | | | |
| phytase | | | | | | | | | | |
| 2.2 FTU/g | 0.00 | 0.00 | 0.10 | 0.02 | 0.00 | 0.12 | 0.04 | 0.84 | 15.09 | 456 |
| DS phytase | | | | | | | | | | |
| 8.8 FTU/g | 0.00 | 0.00 | 0.10 | 0.04 | 0.00 | 0.12 | 0.07 | 0.87 | 15.27 | 474 |
| DS phytase | | | | | | | | | | |
| 22 FTU/g | 0.00 | 0.01 | 0.08 | 0.02 | 0.00 | 0.15 | 0.08 | 0.99 | 15.33 | 471 |
| DS phytase | | | | | | | | | | |

The data in Table 3 is for 1 experiment. However, a number of different experiments were performed and showed that for de-hulled red sorghum, the yield increased from between about 4.5% to about 8.7% in the presence of the phytase.

### Example 2

### Effect of Phytase on Red Sorghum with Hull

Red sorghum from Australia with hull was ground using a FOSS 1093 miller, and then screened by passing through a 30 mesh or 60 mesh screen to obtain 30 mesh or 60 mesh powders. The moisture of the sorghum was 12.42% and the starch content was 64.8%. Sorghum of 27.4 gram was mixed with 92.6 gram of water to make the slurry. Phytase (Danisco US, Inc, Genencor Division) was added to the fermentations in combination with the AA and GA used in Example 1. The control contained no phytase. Fermentations were conducted as in Example 1. The results are shown in Table 4. In the Table the ethanol yield is given with respect to 1 MT sorghum to 95.5% ethanol (L) at 20° C. When using conventional methods to distill ethanol, 95.5% is the maximum amount that can be achieved at 20° C. The abbreviations used in the Table are as follows: Gluc (Glucose); Fruc (Fructose); Suc acid (Succinic acid); Lac acid (Lactic acid); Glyc (Glycerol); Acet acid (Acetic acid); EtOH (ethanol). Samples 1, 2, 5, 6, 9, and 10 were conducted using 60 mesh sorghum. Samples 3, 4, 7, 8, 11, and 12 were conducted using 30 mesh sorghum.

TABLE 4
Effect of Phytase on red sorghum from Australia:

| | | | | | | | % | | | |
|---|---|---|---|---|---|---|---|---|---|---|
| | | | % | % | % | % w/v | w/v | % | | EtOH |
| | % w/v | % w/v | w/v | w/v | w/v | suc | lact | w/v | % v/v | yield/ |
| Samples | DP > 3 | DP-3 | DP-2 | Gluc | Fruc | acid | acid | glyc | EtOH | MT |
| 1# w/out Phytase- | 0.47 | 0.00 | 0.00 | 0.00 | 0.01 | 0.10 | 0.00 | 0.92 | 9.17 | 383 |
| 20% DS | | | | | | | | | | |
| 2# with 44 FTU/g | 0.15 | 0.00 | 0.00 | 0.12 | 0.00 | 0.08 | 0.02 | 0.62 | 9.59 | 403 |
| phytase-20% DS | | | | | | | | | | |
| 3# w/out Phytase- | 0.11 | 0.00 | 0.00 | 0.08 | 0.00 | 0.09 | 0.06 | 0.71 | 9.38 | 376 |
| 20% DS | | | | | | | | | | |
| 4# with 44 FTU/g | 0.12 | 0.00 | 0.00 | 0.12 | 0.00 | 0.09 | 0.04 | 0.71 | 9.75 | 405 |
| phytase-20% | | | | | | | | | | |
| 5# w/out Phytase- | 0.20 | 0.00 | 0.01 | 0.11 | 0.00 | 0.10 | 0.03 | 0.78 | 12.00 | 387 |
| 25% DS | | | | | | | | | | |
| 6# with 44 FTU/g | 0.18 | 0.00 | 0.00 | 0.16 | 0.00 | 0.11 | 0.00 | 0.77 | 12.19 | 397 |
| phytase-25% DS | | | | | | | | | | |
| 7# w/out Phytase- | 0.13 | 0.00 | 0.00 | 0.00 | 0.11 | 0.11 | 0.07 | 0.75 | 11.95 | 385 |
| 25% DS | | | | | | | | | | |
| 8# with 44 FTU/g | 0.12 | 0.00 | 0.00 | 0.00 | 0.14 | 0.15 | 0.09 | 0.91 | 11.51 | 393 |
| phytase-25% DS | | | | | | | | | | |
| 9# w/out Phytase- | 0.25 | 0.00 | 0.05 | 0.14 | 0.00 | 0.11 | 0.09 | 1.00 | 15.07 | 389 |
| 30% DS | | | | | | | | | | |
| 10# with 44 FTU/g | 0.25 | 0.00 | 0.08 | 0.23 | 0.00 | 0.13 | 0.03 | 1.11 | 15.35 | 396 |
| phytase-30% DS | | | | | | | | | | |
| 11# w/out Phytase- | 0.20 | 0.00 | 0.08 | 0.14 | 0.00 | 0.12 | 0.07 | 1.17 | 14.78 | 378 |
| 30% DS | | | | | | | | | | |
| 12# with 44 FTU/g | 0.18 | 0.00 | 0.06 | 0.24 | 0.00 | 0.13 | 0.07 | 1.14 | 15.56 | 389 |
| phytase-30% DS | | | | | | | | | | |

The data in Table 4 is for a single experiment. However, the results of multiple experiments showed that, for hulled red sorghum, the increase in the yield varied, but was typically in the range of about 2.0% to about 7.8%. The above experiment only tested a single dose of phytase.

### Example 3

### Phytase Dosage

To determine the optimal dosage of phytase, sorghum from Example 1 was tested using a range of phytase dosages (from 4.4 FTU/g DS phytase to 44 FTU/g DS phytase). The fermentations were conducted as in Example 1. Table 5 provides the data showing that an increase in the ethanol yield with all dosages, but that 44 FTU/g phytase gave the highest yield. Without being restricted to a specific theory, removal of phosphate groups in phytic acid by phytase produces inositol which has been shown to play a major role in yeast physiology, particularly in the synthesis of structural components of cellular membranes. The effect of inositol on phospholipids, cell growth, ethanol production and ethanol tolerance of Saccharomyces sp., for example, is very beneficial (see e.g., Chi et al. 1999, J. Industrial Micro. and Biotechnol., 22:58-63). This is because the inositol helps synthesis, which results in increased phosphatidylinositol content. Second, high phosphatidylinositol content causes yeast to produce ethanol more rapidly and to tolerate higher concentrations of ethanol. Thus, the breakdown of phytic acid has a number of beneficial effects that result in an increased fermentation efficiency and an increased ethanol yield.

TABLE 5

| | | | 1 MT | 1 MT |
|---|---|---|---|---|
| | Distilled | | sorghum | sorghum |
| Australian | EtOH to | Distilled EtOH | to | to 95.5% |
| sorghum | 20 C. % w/v | (g) | ethanol (L) | EtOH (L) |
| A# w/out | 11.27 | 16.51 | 371.0 | 380.4 |
| phytase | | | | |
| B# + 4.4 FTU | 11.48 | 16.77 | 376.9 | 386.5 |
| phytase | | | | |
| C# + 8.8 FTU | 11.50 | 16.82 | 378.1 | 387.7 |
| phytase | | | | |
| D# + 22 FTU | 11.52 | 16.85 | 378.7 | 388.3 |
| phytase | | | | |
| E# + 44 FTU | 11.76 | 17.21 | 386.7 | 396.5 |
| phytase | | | | |

### Example 4

### Effect of Secondary Enzymes: BLEND F

White sorghum (de-hulled red sorghum) from local supermarkets in Australia was used to identify the effect of secondary enzymes on sorghum. The sorghum was ground using a FOSS 1093 miller, and then strained by passing through a 30 mesh screen. The resulting 30 mesh powders were fermented as in Example 1. Distillation of the fermentation whole broth was carried out for calculating the ethanol yield per metric ton of sorghum. The results are shown in Table 6. In the Table the ethanol yield is given with respect to 1 MT sorghum to 95.5% ethanol (L) at 20° C. When using conventional methods to distill ethanol, 95.5% is the maximum amount that can be achieved at 20° C. The abbreviations used in the Table are as follows: Gluc (Glucose); Fruc (Fructose); Suc acid (Succinic acid); Lac acid (Lactic acid); Glyc (Glycerol); Acet acid (Acetic acid); EtOH (ethanol).

TABLE 6
Effect of BLEND F with and without FERMGEN:

| | | | | | % | % | | | |
|---|---|---|---|---|---|---|---|---|---|
| | | | | % | w/v | w/v | | | EtOH |
| | % w/v | % w/v | % w/v | w/v | suc | lact | % w/v | % v/v | yield/ |
| Enzyme | DP > 3 | DP-2 | Gluc | Fruc | acid | acid | glyc | EtOH | MT |
| STARGEN | 0.12 | 0.12 | 0.12 | 0.05 | 0.13 | 0.09 | 0.86 | 14.30 | 438 |
| 001 control | | | | | | | | | |
| BLEND F | 0.13 | 0.06 | 0.13 | 0.00 | 0.12 | 0.08 | 0.85 | 14.56 | 462 |
| BLEND F + | 0.12 | 0.04 | 0.08 | 0.00 | 0.12 | 0.00 | 0.82 | 14.78 | 472 |
| FERMGEN | | | | | | | | | |

The control, STARGEN 001 is a mixture of AA and GA. BLEND F was a mixture of GSHE alpha amylase (SSU2000), beta-glucosidase (BLGU 160), GSHE glucoamylase (GAU 400) and BP-17 phytase from Buttiauxella sp. (FTU 2500). BLEND F was tested with and without the addition of 3 ppm acid fungal protease (FERMGEN). The results in Table 6 show that when the secondary enzymes were added to the AA and GA, the amount of ethanol produced increased. When the AFP was added to the blend, the amount of ethanol increased as compared to the blend without AFP. Thus, the addition of beta glucosidase and phytase increased the ethanol yield as compared to the AA and GA alone. The % DP-3% w/v was 0 in all cases.

### Example 5

### Phytic Acid Content in DDGS and Thin Stillage

To identify the reduction in phytic acid in the DDGS and thin stillage, the DDGS and thin stillage from Example 2 (red sorghum with hull) were collected and the phytic acid content determined by the nitric thorium assay (see above in Methods section). The results are shown in Table 6. The “Before” fermentation column is for the red sorghum raw material. “w/phytase” means that phytase was included during the fermentation. “w/out phytase” means that phytase was not included during the fermentation. The % refers to the amount of phytic acid w/w dry base (moisture corrected). The cake corresponds to the DDGS.

TABLE 7
Phytic acid in the DDGS

| | Before | After fermentation | After fermentation |
|---|---|---|---|
| | fermentation | Cake | Thin stillage |
| w/phytase | — | 0.29% | 0.14% |
| w/out | 0.59% | 0.56% | 0.17% |
| phytase | | | |

The data in Table 7 showed a large reduction (about 50%) in the amount of phytic acid in the cake. The reduction in the thin stillage was smaller, but still effective in reducing the phytic acid of the thin stillage to be added back to the slurry.

### Example 6

### Fermentation of a Mixed Grain: Sorghum and Corn

Red sorghum from Australia (Enzyme Solutions, Australia) and corn from BBCA (BBCA, China) were ground using a FOSS 1093 miller, and then screened through 30 or 40 mesh respectively. Blends of different ratios of corn and sorghum were made as shown in Table 8.28% and 32% DS slurries were prepared and pHs were adjusted with 26% diluted sulfuric acid. The enzyme formulations in Example 4 were added to the slurry, together with yeast at 0.4% of the dry weight. For the 28% DS slurries, the fermentations were terminated at 67 hrs. For the 32% DS slurries, the fermentations were terminated at 93 hrs. After distillation the whole broth stillage was baked in a 60° C. oven to obtain a dry cake for the dry method of RS analysis. At the end of the fermentation, samples were taken and checked by both HPLC analysis (Table 9) and distillation analysis (Table 10).

Distillation of the fermentation whole broth was carried out for calculating the ethanol yield per metric ton of sorghum. The results are shown in Table 10. In the Table the ethanol yield is given with respect to 1 MT sorghum to 95.5% ethanol (L) at 20° C. When using conventional methods to distill ethanol, 95.5% is the maximum amount that can be achieved at 20° C. The abbreviations used in the Table are as follows: Gluc (Glucose); Fruc (Fructose); Suc acid (Succinic acid); Lac acid (Lactic acid); Glyc (Glycerol); Acet acid (Acetic acid); EtOH (ethanol).

TABLE 8

| | DS | % of | % of BBCA | Particle | GC 004 |
|---|---|---|---|---|---|
| Sample series | (w/w %) | sorghum | corn flour | size | (GAU/g) |
| Australian sorghum (<40 mesh) | 28.0 | 100.0 | 0.0 | <40 | 1.0 |
| 75% Australian sorghum (<40 mesh) + | 28.0 | 75.0 | 25.0 | <40 | 1.0 |
| 25% BBCA corn flour (<40 mesh) | | | | | |
| 50% Australian sorghum (<40 mesh) + | 28.0 | 50.0 | 50.0 | <40 | 1.0 |
| 50% BBCA corn flour (<40 mesh) | | | | | |
| 25% Australian sorghum (<40 mesh) + | 28.0 | 25.0 | 75.0 | <40 | 1.0 |
| 75% BBCA corn flour (<40 mesh) | | | | | |
| BBCA corn flour (<40 mesh) | 28.0 | 0.0 | 0.0 | <40 | 1.0 |
| 50% Australian sorghum (<30 mesh) + | 28.0 | 50.0 | 50.0 | <30 | 1.0 |
| 50% BBCA corn flour (<30 mesh) | | | | | |
| 50% Australian sorghum (<40 mesh) + | 32.0 | 50.0 | 50.0 | <40 | 1.0 |
| 50% BBCA corn flour (<40 mesh) | | | | | |
| 50% Australian sorghum (<30 mesh) + | 32.0 | 50.0 | 50.0 | <30 | 1.0 |
| 50% BBCA corn flour (<30 mesh) | | | | | |

TABLE 9
HPLC results

| | | The calculated results | | | | | | | | | |
|---|---|---|---|---|---|---|---|---|---|---|---|
| | | | | | | | % w/v | | | | |
| | | % w/v | % w/v | % w/v | % w/v | % w/v | succinic | % w/v | % w/v | % w/v | % v/v |
| Local sorghum | time (h) | DP > 3 | DP-3 | DP-2 | Glucose | Fructose | acid | lactic | glycerol | acetic acid | ethanol |
| Australian sorghum (<40 mesh) | 72 | 0.22 | 0.00 | 0.09 | 0.21 | 0.00 | 0.11 | 0.07 | 0.86 | 0.00 | 14.60 |
| 75% Australian sorghum (<40 mesh) + | 72 | 0.22 | 0.00 | 0.07 | 0.22 | 0.00 | 0.14 | 0.09 | 0.98 | 0.00 | 14.51 |
| 25% BBCA corn flour (<40 mesh) | | | | | | | | | | | |
| 50% Australian sorghum (<40 mesh) + | 72 | 0.22 | 0.00 | 0.07 | 0.22 | 0.00 | 0.16 | 0.03 | 1.04 | 0.00 | 14.65 |
| 50% BBCA corn flour (<40 mesh) | | | | | | | | | | | |
| 25% Australian sorghum (<40 mesh) + | 72 | 0.21 | 0.00 | 0.06 | 0.17 | 0.00 | 0.17 | 0.10 | 0.86 | 0.00 | 14.28 |
| 75% BBCA corn flour (<40 mesh) | | | | | | | | | | | |
| BBCA corn flour (<40 mesh) | 72 | 0.23 | 0.00 | 0.06 | 0.21 | 0.00 | 0.15 | 0.10 | 0.89 | 0.00 | 14.58 |
| 50% Australian sorghum (<30 mesh) + | 72 | 0.20 | 0.00 | 0.07 | 0.24 | 0.00 | 0.14 | 0.10 | 0.90 | 0.00 | 14.58 |
| 50% BBCA corn flour (<30 mesh) | | | | | | | | | | | |
| 50% Australian sorghum (<40 mesh) + | 93 | 0.24 | 0.00 | 0.10 | 0.50 | 0.00 | 0.14 | 0.11 | 1.01 | 0.03 | 17.27 |
| 50% BBCA corn flour (<40 mesh) | | | | | | | | | | | |
| 50% Australian sorghum (<30 mesh) + | 93 | 0.21 | 0.00 | 0.10 | 0.38 | 0.02 | 0.14 | 0.10 | 1.04 | 0.00 | 17.01 |
| 50% BBCA corn flour (<30 mesh) | | | | | | | | | | | |

TABLE 10
Distillation and calculation by weight method

| | | | 1MT | Efficiency |
|---|---|---|---|---|
| | Fermentation | Distilled | mixed | by distillation |
| Sample | time | ethanol | grain to | weight |
| name | (hrs) | to 20° C. % (v/v) | ethanol (L) | method (%) |
| Australian sorghum (<40 mesh) | 72 | 13.22 | 383.5 | 92.8 |
| 75% Australian sorghum (<40 mesh) + 25% | 72 | 13.30 | 385.0 | 91.5 |
| BBCA corn flour (<40 mesh) | | | | |
| 50% Australian sorghum (<40 mesh) + 50% | 72 | 13.60 | 393.2 | 91.7 |
| BBCA corn flour (<40 mesh) | | | | |
| 25% Australian sorghum (<40 mesh) + 75% | 72 | 13.47 | 391.1 | 89.6 |
| BBCA corn flour (<40 mesh) | | | | |
| BBCA corn flour (<40 mesh) | 72 | 13.52 | 392.0 | 88.3 |
| 50% Australian sorghum (<30 mesh) + 50% | 72 | 13.45 | 390.1 | 91.0 |
| BBCA corn flour (<30 mesh) | | | | |
| 50% Australian sorghum (<40 mesh) + 50% | 93 | 16.01 | 399.3 | 93.2 |
| BBCA corn flour (<40 mesh) 32% DS | | | | |
| 50% Australian sorghum (<30 mesh) + 50% | 93 | 15.96 | 398.1 | 92.9 |
| BBCA corn flour (<30 mesh) 32% DS | | | | |

The results in Table 9 and Table 10 show that the fermentation process worked well for a mixture of sorghum and corn.

### Example 7

### Comparison of Hot-Cook and No-Cook Processes Using Sorghum

The fermentation efficiency of sorghum in ethanol fermentation of the present invention was then compared with a conventional hot-cook process using STARGEN 001 to produce ethanol from sorghum. The process was compared to a no-cook process using STARGEN 001. The new no-cook process used the blend F from Example 4. Each process is further explained below.

Conventional hot-cook processes involve first milling the sorghum to a specific particle size (<1.0 mm) and then processing without further separating out the various components of the grain. The milled sorghum can be mixed with fresh water and/or thin stillage (10-50% as slurry make up water) and/or condensate water to produce a mash with a dry solids (ds) content ranging from 25% to 45%. The pH can be adjusted to pH 5.8 to 6.0 using dilute sodium hydroxide or ammonia with water, and further subjected to one of the following high temperature liquefaction processes: 1) single dose enzyme addition without jet cooking, 2) Split dose enzyme addition with jet cooking. In a single dose enzyme addition process, a thermostable alpha amylase is added and the slurry is cooked at high temperature, 85-90° C. for a period of 120 to 180° C.time. Then the temperature is then lowered to 32° C. and then pH is reduced to pH less than 5.0 using dilute sulphuric acid prior to fermentation. But in split dose enzyme addition with jet cooking liquefaction process, thermostable alpha amylase is added to the slurry and incubated at 85° C. for 20-45 min and then passed through a jet cooker maintained in the range of 200-225° F. with a hold time of 3 to 5 minutes to complete the gelatinization of the granular starch. The gelatinized starch slurry is then flashed to atmospheric pressure and the temperature maintained at about 85° C. A second dose of thermostable alpha amylase is then added to complete the liquefaction of starch by holding for an additional 90 to 120 minutes. The high temperature also reduces the high risk of microbial contamination of the mash. A bacterial derived thermostable alpha amylases from Bacillus licheniformis or Bacillus stearothennophilus. For example, SPEZYME™ FRED, SPEZYME Xtra (from Danisco, US, Inc, Genencor Division), Termamyl™ SC or Termamyl™ SUPRA from Novozymes) is used to first liquefy the starch at high temperature, >95° C. at pH 5.4-6.5 to a low DE (dextrose equivalent) soluble starch hydrolysate After liquefaction, the pH of the mash is decreased to pH 4.2 to 4.5 using dilute sulfuric acid and then cooled to 32° C. prior to fermentation.

For the comparison of the hot-cook process to the no-cook processes, whole Red sorghum from Australia (12.42% moisture and 64.8 ds) was milled using a FOSS 1093 miller, and then sieved screened through a 30 mesh screen to obtain less than 30 mesh flours. An aliquot of milled sorghum flour (27.4 grams) The ground Sorghum was mixed with 92.6 gram of water to make the slurry containing 24% ds sorghum. no-cook yeast fermentation experiments were conducted using STARGEN™ 001 and the enzyme blend of the present invention, i.e. Blend F from Example 4. In the conventional hot cook process SPEZYME™ XTRA (alpha amylase from Danisco US, Inc, Genencor division) was added to the slurry at dose 0.4 kg/T and the pH was adjusted to 5.6 with 20% sulphuric acid, the mixture was heated up to 110° C. and held for 10 min, then cooled down to 95° C. An additional dose of SPEZYME™ Xtra was added and the liquefaction was continued for another 90 min to complete the hydrolysis. The liquefact was cooled to 32° C. and transferred to a 500 ml Erlenmeyer flask, Glucoamylase (GA-L NEW-Danisco US, Inc, Genencor Division) was added at 1.0 kg/T with active dry yeast (Angel, China) at a dose of 0.4% of dry substance, Urea (Mingfeng, China) was added at 400 ppm for pH. The fermentation was carried out at 32° C. with mild mixing. The fermentation broth at 72° C. was analyzed for ethanol yield using HPLC and distilled in a vacuum evaporator for calculating the ethanol yield per metric ton of sorghum.

TABLE 11
Comparison of the fermentation efficiency of sorghum
of the present invention with Conventional hot-cook
and STARGEN ™ 001 Processes.

| | Ethanol yield, | | |
|---|---|---|---|
| | Fermentation | Ethanol yield | |
| Ethanol | broth 72 hrs, | Liters/MT | % Fermentation |
| Process | % V/V | sorghum | Efficiency |
| Conventional | 11.5 | 375.4 | 90.5 |
| hot-Cook | | | |
| STARGEN ™ 001 | 11.8 | 383.8 | 92.3 |
| no-cook | | | |
| BLEND F | 12.1 | 393.9 | 94.5 |
| no-cook | | | |

Whole sorghum-starch content:-64.8% ds; Moisture content-11.4%.

The data in Table 11 showed a significant increase in the fermentation efficiency of the present invention using the enzyme composition having non-starch hydrolyzing enzymes, phytase and protease together with a glucoamylase (GSHE) and alpha amylase. Both the ethanol yield and the fermentation efficiency were increased when using BLEND F relative to a no-cook process with only AA and GA. Both the ethanol yield and the fermentation efficiency were also increased when using BLEND F relative to a conventional process with AA and GA.

All publications and patents mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention should not be unduly limited to such specific embodiments.

What is claimed is:

1. A method of producing ethanol from sorghum comprising,
contacting a slurry comprising sorghum having a dry solids (ds) content of 20 to 50% w/w with at least one phytase, at least one alpha amylase (AA), at least one glucoamylase (GA), at least one non-starch polysaccharide hydrolyzing enzyme and a fermentation organism for a time sufficient to produce ethanol and at a temperature below the starch gelatinization temperature of sorghum at a pH of about 3.5 to about 7.0 for about 10 to about 250 hours, wherein said at least one AA and/or at least one GA is a granular starch hydrolyzing enzyme (GSHE).

2. The method of claim 1, wherein the at least one non-starch polysaccharide hydrolyzing enzyme is chosen from: a cellulase, a beta-glucosidase, a pectinase, a xylanase, a beta-glucanase and/or a hemicellulase.

3. The method of claim 1, wherein at least two of the: at least one phytase, at least one alpha amylase (AA), at least one glucoamylase (GA), and at least one non-starch polysaccharide hydrolyzing enzyme is added as a blend.

4. The method of claim 1, further comprising contacting the slurry with at least a second non-starch polysaccharide hydrolyzing enzyme.

5. The method of claim 1, further comprising contacting the slurry with at least one protease.

6. The method of claim 5, wherein the at least one protease is an acid fungal protease.

7. The method of claim 6, wherein the acid fungal protease is a Trichoderma acid fungal protease.

8. The method of claim 6, wherein the acid fungal protease is added at a concentration of between about 1 ppm and about 10 ppm.

9. The method of claim 1, wherein the temperature below the gelatinization temperature is 20° C. to 80° C.

10. The method of claim 9, wherein the temperature is 55° C. to 77° C. and the temperature is reduced to 25-20 before the yeast is added.

11. The method of claim 9, wherein the temperature is between about 25° and about 40° C.

12. The method of claim 1, wherein both the AA and the GA is a GSHE.

13. The method of claim 1, wherein the amount of phytase supplied in the contacting step is from about 0.01 to about 10.0 FTU/g ds.

14. The method of claim 13, wherein the amount of phytase supplied in the contacting step is from about 0.1 to about 5.0 FTU/g ds.

15. The method of claim 14, wherein the amount of phytase supplied in the contacting step is from about 1 to about 4 FTU/g ds.

16. The method of claim 1, wherein the slurry comprises sorghum in admixture with at least one other granular starch substrate chosen from corn, wheat, rye, barley, and rice.

17. The method of claim 1, further comprising recovering the ethanol.

18. The method of claim 1, wherein the fermenting organism is a yeast.

19. A process for increasing the yield of ethanol from sorghum, comprising,
obtaining a slurry of sorghum,
contacting the slurry with a combination of enzymes comprising a phytase, an alpha amylase, a glucoamylase, and a non-starch polysaccharide hydrolyzing enzyme to produce fermentable sugars, wherein the alpha amylase and/or the glucoamylase is a GSHE at a temperature below the gelatinization temperature of sorghum; and
fermenting the fermentable sugars in the presence of a fermenting microorganism at a temperature of between 10° C. and 40° C. for a period of 10 hours to 250 hours to produce ethanol, wherein the yield of ethanol is increased relative to a comparable method using only an alpha amylase and a glucoamylase.

20. The process of claim 19, wherein the contacting and fermenting are simultaneous and the temperature is between 10° C. and 40° C.

21. The process of claim 19, wherein the fermenting microorganism is a yeast.

22. The process according to claim 19, wherein the non-starch polysaccharide hydrolyzing enzyme is chosen from: a cellulase, a beta-glucosidase, a pectinase, a xylanase, a beta-glucanase and/or a hemicellulase.

23. The process of claim 19, further comprising contacting the slurry with at least one protease.

24. The process of claim 23, wherein the protease is an acid fungal protease.

25. The process of claim 19, wherein the sorghum is mixed with at least one other grain chosen from: corn, wheat, rye, barley, and rice.

26. The process of claim 19, wherein the ethanol yield is increased at least 4%.

27. The process of claim 19, wherein the ethanol yield is increased between at least 1% and at least 10%.

28. The process of claim 19, wherein the yield is increased relative to a conventional method using sorghum.

29. The process of claim 19, further comprising reducing the temperature after the contacting step and before the fermenting step.

30. A method of producing ethanol from sorghum comprising,
contacting a slurry comprising granular starch with at least one phytase, at least one alpha amylase (AA), at least one glucoamylase (GA) at least one non-starch polysaccharide hydrolyzing enzyme, at least one acid fungal protease and a fermentation organism for a time sufficient to produce ethanol, wherein said at least one AA and/or at least one GA is a granular starch hydrolyzing enzyme (GSHE), at a temperature below the starch gelatinization temperature of sorghum, wherein said non-starch polysaccharide hydrolyzing enzymes are chosen from: a cellulase, a xylanase, a pectinase, a beta-glucosidase, a beta-glucanase and/or a hemicellulase.

31. The method of claim 30, wherein the slurry has a dry solids (ds) content of 20 to 50% w/w.

32. The method of claim 31, wherein the slurry has a dry solids (ds) content of 25-35% w/w.
